(19) United States
(12) Patent Application Publication
(10) Pub. No.: US 2024/0173330 A1
(43) Pub. Date: May 30, 2024

(54) USE OF ATR INHIBITORS IN COMBINATION WITH PARP INHIBITORS FOR TREATING CANCER

(71) Applicant: Repare Therapeutics Inc., St-Laurent (CA)

(72) Inventors: Cameron Black, St-Laurent (CA); Michal Zimmermann, Pointe-Claire (CA)

(21) Appl. No.: 18/540,119

(22) Filed: Dec. 14, 2023

Related U.S. Application Data
(63) Continuation of application No. PCT/CA2022/050970, filed on Jun. 16, 2022.
(60) Provisional application No. 63/336,026, filed on Apr. 28, 2022.
(60) Provisional application No. 63/211,354, filed on Jun. 16, 2021.

(51) Int. Cl. A61K 31/5377; A61K 31/496; A61P 35/00

(52) U.S. Cl. CPC A61K 31/5377; A61K 31/496; A61P 35/00

(57) ABSTRACT

Disclosed are methods of treating a cancer in a subject using an ATR inhibitor and PARP inhibitor. wherein the cancer has been previously identified as a cancer having a loss of function of ATM, BRCA2, RNAse H2A, RNAse H2B, CDK12, or a combination thereof, or as an ALT+ cancer. Also disclosed are methods of inducing cell death in an aberrant cancer cell having a loss of function of ATM, BRCA2, RNAse H2A, RNAse H2B, CDK12, or a combination thereof, or an ALT+ cancer cell, by contacting the cell with an effective amount of an ATR inhibitor and PARP inhibitor. The PARP inhibitor may be a compound of formula (III) or (IV):

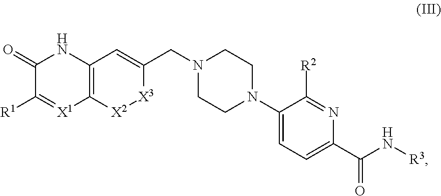

where
X¹ and X² are each independently selected from N and C(H),
X³ is independently selected from N and C(R⁴), wherein R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹═C(H), and X³ is C(R⁴), and
when X³ is N, then X¹ and X² are both C(H); or

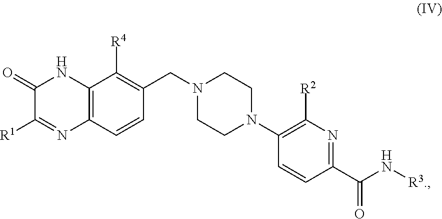

where
R¹ is independently selected from H, C₁₋₄ alkyl, C₃₋₆ cycloalkyl, C₁₋₄ fluoroalkyl, and C₁₋₄ alkyloxy;
R² is independently selected from H, halo, C₁₋₄ alkyl, and C₁₋₄ fluoroalkyl;
R³ is H or C₁₋₄ alkyl; and
R⁴ is halo or C₁₋₄ alkyl,
or a pharmaceutically acceptable salt thereof.

### CROSS REFERENCE TO RELATED APPLICATIONS

This application is a Continuation Application of International Application No. PCT/CA2022/050970, filed Jun. 16, 2022, which claims priority to U.S. Provisional Application No. 63/336,026, filed Apr. 28, 2022 and U.S. Provisional Application No. 63/211,354, filed Jun. 16, 2021, the disclosures of which are incorporated herein by reference in their entireties.

### FIELD OF THE INVENTION

The invention relates to combinations of Ataxia-telangiectasia and RAD-3-related protein (ATR) kinase inhibitors, pharmaceutically acceptable salts thereof, or pharmaceutical composition containing the same, and Poly (ADP ribose) polymerase (PARP) inhibitors, pharmaceutically acceptable salts thereof, or pharmaceutical composition containing the same, and their use in the treatment of a disease or condition, such as cancer.

### BACKGROUND

DNA damage occurs continually in cells as a result of environmental insults including ultraviolet radiation, X-rays and endogenous stress factors, such as reactive oxygen and hydrolysis of bases. Cancer cells are subject to a higher rate of DNA damage inherently induced by higher rates of DNA replication in these cells. Several DNA damage response (DDR) pathways have evolved in a highly coordinated manner to help repair DNA damage and to act as a cellular checkpoint to stop the replication of cells with damaged DNA, allowing for repair functions to occur before the damaged DNA is passed on to daughter cells. Each of the identified DNA repair pathways sense and repair distinct but overlapping types of DNA damage.

One major DDR protein that acts as a key cell cycle checkpoint is the ataxia telangiectasia mutated and rad3-related (ATR) kinase, related to the family of phosphoinositide 3-kinase-related protein kinases (PIKKs). ATR is activated by single stranded (ss) DNA lesions caused by stalled replication forks or during nucleotide excision repair but is also activated by double strand breaks following DNA end resection during homologous recombination. ATR is recruited to sites of DNA damage by binding to the RPA protein that coats ssDNA along with an accessory factor called ATR-interacting protein (ATRIP). The ATR/ATRIP complex is then activated by recruitment of additional factors in the 9-1-1 complex (RAD 9, RAD1, and HUS1) which subsequently recruits the TOPBP1 protein and represents critical steps for activation of the downstream phosphorylation cascade that results in cell cycle arrest. The primary target for ATR kinase is CHK1, which when phosphorylated, targets both cdc25 proteins and Wee1 resulting in inhibition of cyclin-dependent kinase activity and cell cycle arrest in S-phase or in G2/M.

ATR has been identified as an important cancer target since it is essential for dividing cells. ATR deficient mice are embryonic lethal, however, adult mice with conditional ATR knocked out are viable with effects on rapidly proliferating tissues and stem cell populations. Mouse embryonic stem cells lacking ATR will only divide for 1-2 doublings and then die, suggesting that ATR is required for the maintenance of dividing cells. Interestingly, mice harboring hypomorphic ATR mutations that reduce expression of ATR to 10% of normal levels showed reduced H-rasG12D-induced tumor growth with minimal effects on proliferating normal cells, e.g., the bone marrow or intestinal epithelial cells. Cancer cells that have high levels of replication stress due to oncogenic mutations, dysfunctional G1/S checkpoint control (e.g., loss of p53 function), defects in other DNA repair pathways (e.g., ATM) or that are subject to the effects of DNA damaging agents, e.g., radiation therapy or chemotherapeutic agents, are therefore more dependent on ATR for DNA repair and survival. Together, these results highlight a rationale for the selective sensitivity of proliferating tumor cells to ATR inhibition and the potential for a therapeutic window over healthy proliferating cells.

Inhibitors of poly(ADP-ribose) polymerases (PARP inhibitors) target the DNA repair enzyme poly(ADP-ribose) polymerase 1 (PARP1) and closely related paralogs. Several PARP inhibitors (olaparib, niraparib, rucaparib, talazoparib) have been approved for treatment of various cancers (e.g., ovarian cancer, breast cancer, fallopian tube cancer, and primary peritoneal cancer).

There is a need for new anti-cancer therapies and, in particular, effective combinations of known inhibitors with different mechanisms of action that can synergize to increase overall efficacy and treat a broader spectrum of cancers than either inhibitor alone.

### SUMMARY OF THE INVENTION

In general, the invention provides a combination of an ATR inhibitor, or a pharmaceutically acceptable salt thereof, and a PARP inhibitor, or a pharmaceutically acceptable salt thereof for the treatment of cancers or for inducing cell death in cancer cells. The cancers included herein may be, e.g., cancers having a loss of function of ATM, BRCA2, RNAse H2A, RNAse H2B, CDK12, or a combination thereof. The cancer may be, e.g., an ALT+ cancer. The PARP inhibitor may be a compound of formula (III) or (IV):

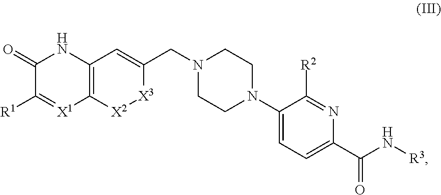

where
X¹ and X² are each independently selected from N and C(H),
X³ is independently selected from N and C(R⁴), where R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹ is C(H), and X³ is C(R⁴), and
when X³ is N, then X¹ and X² are both C(H);
   or

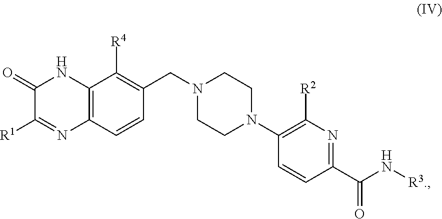

where
R¹ is independently selected from H, C₁₋₄ alkyl, C₃₋₆ cycloalkyl, C₁₋₄ fluoroalkyl, and C₁₋₄ alkyloxy;
R² is independently selected from H, halo, C₁₋₄ alkyl, and C₁₋₄ fluoroalkyl;
R³ is H or C₁₋₄ alkyl; and
R⁴ is halo or C₁₋₄ alkyl,
or a pharmaceutically acceptable salt thereof.

In one aspect, the invention provides a method of treating a cancer in a subject, the method comprising administering to the subject in need thereof a therapeutically effective amount of an ATR inhibitor and PARP inhibitor, where the cancer has been previously identified as a cancer having a loss of function of ATM, BRCA2, RNAse H2A, RNAse H2B, CDK12, or a combination thereof, or the cancer has been previously identified as an ALT+ cancer.

In another aspect, the invention provides a method of treating a cancer in a subject, the method comprising administering to the subject in need thereof a therapeutically effective amount of an ATR inhibitor and PARP inhibitor, where the cancer has a loss of function of ATM serine/threonine kinase, BRCA2, RNAse H2A, RNAse H2B, CDK12, or a combination thereof, or the cancer is an ALT+ cancer.

In yet another aspect, the invention provides a method of treating a cancer in a subject, the method comprising:
(i) identifying the cancer as having a loss of function of ATM, BRCA2, RNAse H2A, RNAse H2B, CDK12, or a combination thereof, or as an ALT+ cancer; and
(ii) administering to the subject in need thereof a therapeutically effective amount of an ATR inhibitor and PARP inhibitor.

In some embodiments, the ATR inhibitor is administered before the PARP inhibitor (e.g., within 1 week, within 6 days, within 5 days, within 4 days, within 3 days, within 2 days, within 1 day, or within 12 hours). In some embodiments, the ATR inhibitor is administered after the PARP inhibitor (e.g., within 1 week, within 6 days, within 5 days, within 4 days, within 3 days, within 2 days, within 1 day, or within 12 hours). In some embodiments, the ATR inhibitor is co-administered with the PARP inhibitor. In some embodiments, the ATR inhibitor is administered intermittently (e.g., 1 day/week, 2 days/week, or 3 days/week). In some embodiments, the PARP inhibitor is administered daily, 1 day/week, 2 days/week, 3 days/week, or 4 days/week. In some embodiments, the PARP inhibitor is administered 1 day/week, 2 days/week, 3 days/week, or 4 days/week. In some embodiments, the PARP inhibitor is administered on a continuous daily basis.

In some embodiments, the therapeutically effective amount is a subtherapeutic regimen of the ATR inhibitor. In some embodiments, the therapeutically effective amount is a subtherapeutic regimen of the PARP inhibitor. In some embodiments, the subtherapeutic regimen comprises a starting dosage that is at least 50% less than the lowest standard starting dosage that is used for a monotherapy. In some embodiments, the subtherapeutic regimen comprises a maintenance dosage that is at least 50% less than the lowest standard maintenance dosage that is used for a monotherapy. In some embodiments, the maintenance dosage comprises a first reduced dosage. In some embodiments, the maintenance dosage comprises a second reduced dosage. In some embodiments, the maintenance dosage comprises a third reduced dosage. In some embodiments, the route of administration is an oral administration.

In still another aspect, the invention provides a method of inducing cell death in an aberrant cancer cell having a loss of function of ATM, BRCA2, RNAse H2A, RNAse H2B, CDK12, or a combination thereof, or in an ALT+ cancer cell, the method comprising contacting the cell with an effective amount of an ATR inhibitor and an effective amount of a PARP inhibitor, the effective amounts being sufficient to induce cell death in the aberrant cancer cell.

In some embodiments, the loss of function is a loss of function of ATM. In some embodiments, the loss of function is a loss of function of RNAse H2A. In some embodiments, the loss of function is a loss of function of RNAse H2B. In some embodiments, the loss of function is a loss of function of CDK12. In some embodiments, the loss of function is a loss of function of BRCA2. In some embodiments, the cancer is an ALT+ cancer.

In some embodiments, the ATR inhibitor is a compound of formula (I):

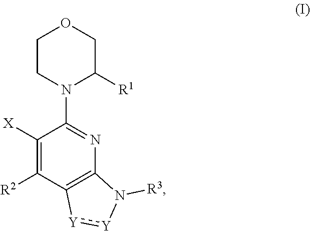

or a pharmaceutically acceptable salt thereof,
where
[symbol] is a double bond, and each Y is independently N or CR⁴; or [symbol] is a single bond, and each Y is independently NR^{Y}, carbonyl, or C(R^{Y})₂; where each R^{Y} is independently H or optionally substituted C₁₋₆ alkyl;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

In some embodiments, the ATR inhibitor is a compound of formula (II):

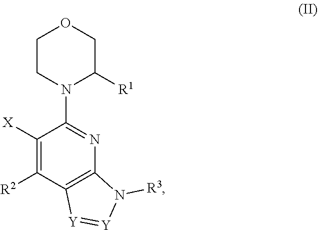

or a pharmaceutically acceptable salt thereof,
where
each Y is independently N or CR⁴;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

In some embodiments, the ATR inhibitor is selected from the group consisting of compounds 43, 57, 62, 87, 93, 94, 95, 99, 100, 106, 107, 108, 109, 111, 112, 113, 114, 115, 116, 118, 119, 120, 121, 122, 123, 135, 147, 148, and pharmaceutically acceptable salts thereof.

In some embodiments, the ATR inhibitor is compound 43 or a pharmaceutically acceptable salt thereof. In some embodiments, the ATR inhibitor is compound 121 or a pharmaceutically acceptable salt thereof. In some embodiments, the ATR inhibitor is compound 122 or a pharmaceutically acceptable salt thereof. In some embodiments, the cancer is renal cell carcinoma, mature B-cell neoplasms, endometrial cancer, ovarian cancer, fallopian tube cancer, primary peritoneal cancer, colorectal cancer, skin cancer, small bowel cancer, non-small cell lung cancer, melanoma, bladder cancer, pancreatic cancer, head and neck cancer, mesothelioma, glioma, prostate cancer, breast cancer, or esophagogastric cancer.

In some embodiments, the PARP inhibitor is a compound of formula (III) or a pharmaceutically acceptable salt thereof. In some embodiments, R³ in formula (III) is C₁₋₄ alkyl. In some embodiments, R³ in formula (III) is methyl. In some embodiments, R¹ in formula (III) is ethyl. In some embodiments, the PARP inhibitor is a compound of formula (IIIa):

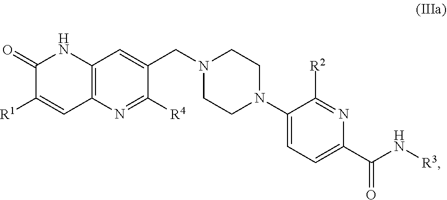

where
R¹ is C₁₋₄ alkyl,
R² is H, halo, C₁₋₄ alkyl, or C₁₋₄ fluoroalkyl,
R³ is H or C₁₋₄ alkyl, and
R⁴ is H,
or a pharmaceutically acceptable salt thereof.

In some embodiments, R² is difluoromethyl, trifluoromethyl, or methyl. In some embodiments, R² is H or halo. In some embodiments, R¹ is ethyl; R² is H, chloro or fluoro; and R³ is methyl.

In some embodiments, the PARP inhibitor is a compound of formula (IIIb):

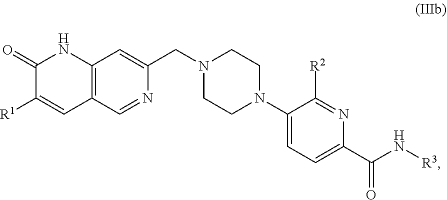

where
R¹ is C₁₋₄ alkyl,
R² is H or halo, and
R³ is H or C₁₋₄ alkyl,
or a pharmaceutically acceptable salt thereof.

In some embodiments, R¹ is ethyl; R² is H, chloro, or fluoro; and R³ is methyl.

In some embodiments, the PARP inhibitor is a compound of formula (IIIc):

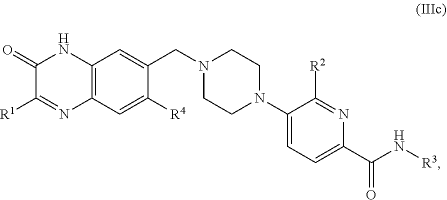

where
R¹ is C₁₋₄ alkyl or C₁₋₄ fluoroalkyl,
R² is H, halo, C₁₋₄ alkyl, or C₁₋₄ fluoroalkyl,
R³ is H or C₁₋₄ alkyl, and
R⁴ is H or fluoro,
or a pharmaceutically acceptable salt thereof.

In some embodiments, R¹ is ethyl, n-propyl, trifluoromethyl, 1,1-difluoroethyl, 2,2-difluroethyl, 2-fluoroethyl, or 2,2,2-trifluoroethyl. In some embodiments, R² is H, methyl, ethyl, trifluoromethyl, difluoromethyl, fluoromethyl, fluoro, or chloro. In some embodiments, R³ is H or methyl. In some embodiments, R⁴ is H.

In some embodiments, the PARP inhibitor is:
5-[4-[(3-ethyl-2-oxo-1H-1,6-naphthyridin-7-yl)methyl]piperazin-1-yl]-N-methyl-pyridine-2-carboxamide,
5-[4-[(3-ethyl-2-oxo-1H-1,6-naphthyridin-7-yl)methyl]piperazin-1-yl]-6-fluoro-N-methyl-pyridine-2-carboxamide,
6-chloro-5-[4-[(3-ethyl-2-oxo-1H-1,6-naphthyridin-7-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-6-fluoro-N-methyl-pyridine-2-carboxamide,
6-chloro-5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]pyridine-2-carboxamide,
6-ethyl-5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-N-methyl-6-(trifluoromethyl)pyridine-2-carboxamide,
6-(difluoromethyl)-5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-6-fluoro-N-methyl-pyridine-2-carboxamide,
5-[4-[(2-ethyl-3-oxo-4H-quinoxalin-6-yl)methyl]piperazin-1-yl]-N,6-dimethyl-pyridine-2-carboxamide,
6-chloro-5-[4-[(2-ethyl-3-oxo-4H-quinoxalin-6-yl)methyl]piperazin-1-yl]-N-methyl-pyridine-2-carboxamide,
N-methyl-5-[4-[[3-oxo-2-(trifluoromethyl)-4H-quinoxalin-6-yl]methyl]piperazin-1-yl]pyridine-2-carboxamide,
6-chloro-N-methyl-5-[4-[[3-oxo-2-(trifluoromethyl)-4H-quinoxalin-6-yl]methyl]piperazin-1-yl]pyridine-2-carboxamide,
6-fluoro-N-methyl-5-[4-[[3-oxo-2-(trifluoromethyl)-4H-quinoxalin-6-yl]methyl]piperazin-1-yl]pyridine-2-carboxamide,
N-methyl-5-[4-[(3-oxo-2-propyl-4H-quinoxalin-6-yl)methyl]piperazin-1-yl]pyridine-2-carboxamide,
6-chloro-N-methyl-5-[4-[(3-oxo-2-propyl-4H-quinoxalin-6-yl)methyl]piperazin-1-yl]pyridine-2-carboxamide,
6-fluoro-N-methyl-5-[4-[(3-oxo-2-propyl-4H-quinoxalin-6-yl)methyl]piperazin-1-yl]pyridine-2-carboxamide,
5-[4-[(2-ethyl-7-fluoro-3-oxo-4H-quinoxalin-6-yl)methyl]piperazin-1-yl]-6-fluoro-N-methyl-pyridine-2-carboxamide,
5-[4-[[2-(1,1-difluoroethyl)-3-oxo-4H-quinoxalin-6-yl]methyl]piperazin-1-yl]-N-methyl-pyridine-2-carboxamide,
5-[4-[[2-(2,2-difluoroethyl)-3-oxo-4H-quinoxalin-6-yl]methyl]piperazin-1-yl]-N-methyl-pyridine-2-carboxamide,
5-[4-[[2-(2,2-difluoroethyl)-3-oxo-4H-quinoxalin-6-yl]methyl]piperazin-1-yl]-6-fluoro-N-methyl-pyridine-2-carboxamide,
5-[4-[[2-(2-fluoroethyl)-3-oxo-4H-quinoxalin-6-yl]methyl]piperazin-1-yl]-N-methyl-pyridine-2-carboxamide,
6-fluoro-5-[4-[[2-(2-fluoroethyl)-3-oxo-4H-quinoxalin-6-yl]methyl]piperazin-1-yl]-N-methyl-pyridine-2-carboxamide,
N-methyl-5-[4-[[3-oxo-2-(2,2,2-trifluoroethyl)-4H-quinoxalin-6-yl]methyl]piperazin-1-yl]pyridine-2-carboxamide,
6-fluoro-N-methyl-5-(4-((3-oxo-2-(2,2,2-trifluoroethyl)-3,4-dihydroquinoxalin-6-yl)methyl) piperazin-1-yl)picolinamide,
   or a pharmaceutically acceptable salt thereof.

In some embodiments, the PARP inhibitor is 5-[4-[(7-ethyl-6-oxo-5H-1,5-naphthyridin-3-yl)methyl]piperazin-1-yl]-N-methyl-pyridine-2-carboxamide, or a pharmaceutically acceptable salt thereof.

In some embodiments, the PARP inhibitor is:
6-(difluoromethyl)-5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-N-methyl-6 (trifluoromethyl)pyridine-2-carboxamide,
5-[4-[(7-ethyl-6-oxo-5H-1,5-naphthyridin-3-yl)methyl]piperazin-1-yl]-N,6-dimethyl-pyridine-2-carboxamide,
N-ethyl-5-[4-[(7-ethyl-6-oxo-5H-1,5-naphthyridin-3-yl)methyl]piperazin-1-yl]pyridine-2-carboxamide,
   or a pharmaceutically acceptable salt thereof.

In some embodiments, the PARP inhibitor is 5-[4-[(7-ethyl-6-oxo-5H-1,5-naphthyridin-3-yl)methyl]piperazin-1-yl]-N-methyl-pyridine-2-carboxamide or a pharmaceutically acceptable salt thereof.

In some embodiments, the PARP inhibitor is a compound of formula (IV) or a pharmaceutically acceptable salt thereof.

In some embodiments, R¹ is methyl, ethyl, isopropyl, cyclopropyl, 1,1-difluoroethyl, 1-fluoroethyl, trifluoromethyl, difluoromethyl, or methoxy. In some embodiments, R¹ is methyl or ethyl.

In some embodiments, R² is H, chloro, fluoro, methyl, or difluoromethyl. In some embodiments, R² is fluoro or methyl.

In some embodiments, R³ is methyl or ethyl.

In some embodiments, R⁴ is chloro, fluoro, or methyl. In some embodiments, R⁴ is fluoro.

In some embodiments, R¹ is C₁₋₄ alkyl, R² is halo, R³ is C₁₋₄ alkyl, and R⁴ is halo or C₁₋₄ alkyl.

In some embodiments, the PARP inhibitor is:
5-[4-[(2,5-dimethyl-3-oxo-4H-quinoxalin-6-yl)methyl]piperazin-1-yl]-6-fluoro-N-methyl-pyridine-2-carboxamide, 5-[4-[(2,5-dimethyl-3-oxo-4H-quinoxalin-6-yl)methyl]piperazin-1-yl]-N-methyl-pyridine-2-carboxamide,
6-chloro-5-[4-[(2,5-dimethyl-3-oxo-4H-quinoxalin-6-yl)methyl]piperazin-1-yl]-N-methyl-pyridine-2-carboxamide,
5-[4-[(2,5-dimethyl-3-oxo-4H-quinoxalin-6-yl)methyl]piperazin-1-yl]-N,6-dimethyl-pyridine-2-carboxamide,
5-[4-[(2,5-dimethyl-3-oxo-4H-quinoxalin-6-yl)methyl]piperazin-1-yl]-6-fluoro-pyridine-2-carboxamide,
5-[4-[(5-fluoro-2-methyl-3-oxo-4H-quinoxalin-6-yl)methyl]piperazin-1-yl]-6-methyl-pyridine-2-carboxamide,
5-[4-[(2,5-dimethyl-3-oxo-4H-quinoxalin-6-yl)methyl]piperazin-1-yl]-6-methyl-pyridine-2-carboxamide,
6-chloro-5-[4-[(5-chloro-2-ethyl-3-oxo-4H-quinoxalin-6-yl)methyl]piperazin-1-yl]-N-methyl-pyridine-2-carboxamide,
5-[4-[(5-chloro-2-ethyl-3-oxo-4H-quinoxalin-6-yl)methyl]piperazin-1-yl]-6-fluoro-N-methyl-pyridine-2-carboxamide,
5-[4-[(5-chloro-2-ethyl-3-oxo-4H-quinoxalin-6-yl)methyl]piperazin-1-yl]-N-methyl-pyridine-2-carboxamide,
5-[4-[(5-chloro-2-ethyl-3-oxo-4H-quinoxalin-6-yl)methyl]piperazin-1-yl]-N,6-dimethyl-pyridine-2-carboxamide,
6-fluoro-5-[4-[[5-fluoro-2-[(1S and 1R)-1-fluoroethyl]-3-oxo-4H-quinoxalin-6-yl]methyl]piperazin-1-yl]-N-methyl-pyridine-2-carboxamide,
5-[4-[[5-fluoro-2-[(1S and 1R)-1-fluoroethyl]-3-oxo-4H-quinoxalin-6-yl]methyl]piperazin-1-yl]-N,6-dimethyl-pyridine-2-carboxamide,
5-[4-[(5-chloro-2-methyl-3-oxo-4H-quinoxalin-6-yl)methyl]piperazin-1-yl]-N-methyl-pyridine-2-carboxamide,
5-[4-[(5-chloro-2-methyl-3-oxo-4H-quinoxalin-6-yl)methyl]piperazin-1-yl]-6-fluoro-N-methyl-pyridine-2-carboxamide,
5-[4-[(5-chloro-2-methyl-3-oxo-4H-quinoxalin-6-yl)methyl]piperazin-1-yl]-N,6-dimethyl-pyridine-2-carboxamide,
5-[4-[[2-(1,1-difluoroethyl)-5-fluoro-3-oxo-4H-quinoxalin-6-yl]methyl]piperazin-1-yl]-N, 6-dimethyl-pyridine-2-carboxamide,
6-fluoro-5-[4-[(5-fluoro-2-methyl-3-oxo-4H-quinoxalin-6-yl)methyl]piperazin-1-yl]-N-methyl-pyridine-2-carboxamide, 6-(difluoromethyl)-5-[4-[(5-fluoro-2-methyl-3-oxo-4H-quinoxalin-6-yl)methyl]piperazin-1-yl]-N-methyl-pyridine-2-carboxamide,
6-fluoro-5-[4-[(5-fluoro-2-methyl-3-oxo-4H-quinoxalin-6-yl)methyl]piperazin-1-yl]pyridine-2-carboxamide,
5-[4-[(2-ethyl-5-fluoro-3-oxo-4H-quinoxalin-6-yl)methyl]piperazin-1-yl]-N,6-dimethyl-pyridine-2-carboxamide,
6-(difluoromethyl)-5-[4-[(2-ethyl-5-fluoro-3-oxo-4H-quinoxalin-6-yl)methyl]piperazin-1-yl]-N-methyl-pyridine-2-carboxamide,
5-[4-[(2-ethyl-5-fluoro-3-oxo-4H-quinoxalin-6-yl)methyl]piperazin-1-yl]pyridine-2-carboxamide,
5-[4-[(2-ethyl-5-fluoro-3-oxo-4H-quinoxalin-6-yl)methyl]piperazin-1-yl]-6-methyl-pyridine-2-carboxamide,
5-[4-[(2-ethyl-5-fluoro-3-oxo-4H-quinoxalin-6-yl)methyl]piperazin-1-yl]-6-fluoro-N-methyl-pyridine-2-carboxamide,
6-chloro-5-[4-[(2-ethyl-5-fluoro-3-oxo-4H-quinoxalin-6-yl)methyl]piperazin-1-yl]-N-methyl-pyridine-2-carboxamide,
5-[4-[(2-ethyl-5-fluoro-3-oxo-4H-quinoxalin-6-yl)methyl]piperazin-1-yl]-N-methyl-pyridine-2-carboxamide,
6-chloro-5-[4-[(5-fluoro-2-methyl-3-oxo-4H-quinoxalin-6-yl)methyl]piperazin-1-yl]-N-methyl-pyridine-2-carboxamide,
5-[4-[(5-fluoro-2-methyl-3-oxo-4H-quinoxalin-6-yl)methyl]piperazin-1-yl]-N,6-dimethyl-pyridine-2-carboxamide,
5-[4-[(5-fluoro-2-methyl-3-oxo-4H-quinoxalin-6-yl)methyl]piperazin-1-yl]-N-methyl-pyridine-2-carboxamide,
5-[4-[(5-fluoro-3-oxo-4H-quinoxalin-6-yl)methyl]piperazin-1-yl]-N-methyl-pyridine-2-carboxamide,
6-chloro-5-[4-[(5-fluoro-3-oxo-4H-quinoxalin-6-yl)methyl]piperazin-1-yl]-N-methyl-pyridine-2-carboxamide,
5-[4-[(5-fluoro-3-oxo-4H-quinoxalin-6-yl)methyl]piperazin-1-yl]-N,6-dimethyl-pyridine-2-carboxamide,
6-fluoro-5-[4-[(5-fluoro-3-oxo-4H-quinoxalin-6-yl)methyl]piperazin-1-yl]-N-methyl-pyridine-2-carboxamide,
5-[4-[[2-(difluoromethyl)-5-fluoro-3-oxo-4H-quinoxalin-6-yl]methyl]piperazin-1-yl]-N, 6-dimethyl-pyridine-2-carboxamide, 5-[4-[(5-fluoro-2-methoxy-3-oxo-4H-quinoxalin-6-yl)methyl]piperazin-1-yl]-N-methyl-pyridine-2-carboxamide,
6-fluoro-5-[4-[(5-fluoro-2-methoxy-3-oxo-4H-quinoxalin-6-yl)methyl]piperazin-1-yl]-N-methyl-pyridine-2-carboxamide,
5-[4-[(5-fluoro-2-methoxy-3-oxo-4H-quinoxalin-6-yl)methyl]piperazin-1-yl]-N,6-dimethyl-pyridine-2-carboxamide,
6-chloro-5-[4-[(5-fluoro-2-methoxy-3-oxo-4H-quinoxalin-6-yl)methyl]piperazin-1-yl]-N-methyl-pyridine-2-carboxamide,
5-[4-[(2-ethyl-5-methyl-3-oxo-4H-quinoxalin-6-yl)methyl]piperazin-1-yl]-N,6-dimethyl-pyridine-2-carboxamide,
5-[4-[(2-ethyl-5-methyl-3-oxo-4H-quinoxalin-6-yl)methyl]piperazin-1-yl]-6-fluoro-N-methyl-pyridine-2-carboxamide,
5-[4-[(2-ethyl-5-methyl-3-oxo-4H-quinoxalin-6-yl)methyl]piperazin-1-yl]-N-methyl-pyridine-2-carboxamide,
N-ethyl-6-fluoro-5-[4-[(5-fluoro-2-methyl-3-oxo-4H-quinoxalin-6-yl)methyl]piperazin-1-yl]pyridine-2-carboxamide,
N-ethyl-5-[4-[(5-fluoro-2-methyl-3-oxo-4H-quinoxalin-6-yl)methyl]piperazin-1-yl]-6-methyl-pyridine-2-carboxamide,
5-[4-[[5-fluoro-3-oxo-2-(trifluoromethyl)-4H-quinoxalin-6-yl]methyl]piperazin-1-yl]-N, 6-dimethyl-pyridine-2-carboxamide,
6-fluoro-5-[4-[[5-fluoro-3-oxo-2-(trifluoromethyl)-4H-quinoxalin-6-yl]methyl]piperazin-1-yl]-N-methyl-pyridine-2-carboxamide,
6-chloro-5-[4-[[5-fluoro-3-oxo-2-(trifluoromethyl)-4H-quinoxalin-6-yl]methyl]piperazin-1-yl]-N-methyl-pyridine-2-carboxamide,
5-[4-[[5-fluoro-3-oxo-2-(trifluoromethyl)-4H-quinoxalin-6-yl]methyl]piperazin-1-yl]-N-methyl-pyridine-2-carboxamide,
6-fluoro-5-[4-[(5-fluoro-2-isopropyl-3-oxo-4H-quinoxalin-6-yl)methyl]piperazin-1-yl]-N-methyl-pyridine-2-carboxamide,
5-[4-[(5-fluoro-2-isopropyl-3-oxo-4H-quinoxalin-6-yl)methyl]piperazin-1-yl]-N,6-dimethyl-pyridine-2-carboxamide,
5-[4-[(5-fluoro-2-isopropyl-3-oxo-4H-quinoxalin-6-yl)methyl]piperazin-1-yl]-N-methyl-pyridine-2-carboxamide, 5-[4-[(2-cyclopropyl-5-fluoro-3-oxo-4H-quinoxalin-6-yl)methyl]piperazin-1-yl]-6-fluoro-N-methyl-pyridine-2-carboxamide,
5-[4-[(2-cyclopropyl-5-fluoro-3-oxo-4H-quinoxalin-6-yl)methyl]piperazin-1-yl]-N,6-dimethyl-pyridine-2-carboxamide,
5-[4-[(2-cyclopropyl-5-fluoro-3-oxo-4H-quinoxalin-6-yl)methyl]piperazin-1-yl]-N-methyl-pyridine-2-carboxamide,
5-[4-[(2-methoxy-5-methyl-3-oxo-4H-quinoxalin-6-yl)methyl]piperazin-1-yl]-N,6-dimethyl-pyridine-2-carboxamide,
6-fluoro-5-[4-[(2-methoxy-5-methyl-3-oxo-4H-quinoxalin-6-yl)methyl]piperazin-1-yl]-N-methyl-pyridine-2-carboxamide,
6-(difluoromethyl)-5-[4-[(2-methoxy-5-methyl-3-oxo-4H-quinoxalin-6-yl)methyl]piperazin-1-yl]-N-methyl-pyridine-2-carboxamide,
6-(difluoromethyl)-5-[4-[(2,5-dimethyl-3-oxo-4H-quinoxalin-6-yl)methyl]piperazin-1-yl]-N-methyl-pyridine-2-carboxamide, or
   a pharmaceutically acceptable salts thereof.

In some embodiments, the PARP inhibitor is 6-fluoro-5-[4-[(5-fluoro-2-methyl-3-oxo-4H-quinoxalin-6-yl)methyl]piperazin-1-yl]-N-methyl-pyridine-2-carboxamide, or a pharmaceutically acceptable salt thereof.

In some embodiments, the PARP inhibitor is 6-fluoro-5-[4-[(5-fluoro-2-methyl-3-oxo-4H-quinoxalin-6-yl)methyl]piperazin-1-yl]-N-methyl-pyridine-2-carboxamide.

In another aspect, the invention provides a method of treating a cancer in a subject, the method comprising administering to the subject in need thereof a therapeutically effective amount of an ATR inhibitor and a therapeutically effective amount of a PARP inhibitor, where the cancer has been previously identified as a cancer having a loss of function of ATM, BRCA2, RNAse H2A, RNAse H2B, CDK12, or a combination thereof, or where the cancer has been previously identified as an ALT+ cancer, and where the PARP inhibitor is veliparib (ABT-888), iniparib (BSI-201), 2X-121, CEP-9722, KU-0059436 (AZD2281), PF-01367338, a pharmaceutically acceptable salt thereof, or a combination thereof.

In yet another aspect, the invention provides a method of treating a cancer in a subject, the method comprising administering to the subject in need thereof a therapeutically effective amount of an ATR inhibitor and a therapeutically effective amount of a PARP inhibitor, where the cancer has a loss of function of ATM, BRCA2, RNAse H2A, RNAse H2B, CDK12, or a combination thereof, or where the cancer is an ALT+ cancer; and where the PARP inhibitor is veliparib (ABT-888), iniparib (BSI-201), 2X-121, CEP-9722, KU-0059436 (AZD2281), PF-01367338, a pharmaceutically acceptable salt thereof, or a combination thereof.

In still another aspect, the invention provides a method of treating a cancer in a subject, the method comprising the steps of
(i) identifying the cancer as having a loss of function of ATM, BRCA2, RNAse H2A, RNAse H2B, CDK12, or a combination thereof, or as an ALT+ cancer; and
(ii) administering to the subject in need thereof a therapeutically effective amount of an ATR inhibitor and a therapeutically effective amount of a PARP inhibitor that is veliparib (ABT-888), iniparib (BSI-201), 2X-121, CEP-9722, KU-0059436 (AZD2281), PF-01367338, a pharmaceutically acceptable salt thereof, or a combination thereof.

In some embodiments, the ATR inhibitor is administered before the PARP inhibitor. In some embodiments, the ATR inhibitor is administered after the PARP inhibitor. In some embodiments, the ATR inhibitor is co-administered with the PARP inhibitor.

In some embodiments, the therapeutically effective amount is a subtherapeutic regimen of the ATR inhibitor. In some embodiments, the therapeutically effective amount is a subtherapeutic regimen of the PARP inhibitor. In some embodiments, the subtherapeutic regimen comprises a starting dosage that is at least 50% less than the lowest standard starting dosage that is used for a monotherapy. In some embodiments, the subtherapeutic regimen comprises a maintenance dosage that is at least 50% less than the lowest standard maintenance dosage that is used for a monotherapy.

In some embodiments, the maintenance dosage comprises a first reduced dosage. In some embodiments, the maintenance dosage comprises a second reduced dosage. In some embodiments, the maintenance dosage comprises a third reduced dosage.

In some embodiments, the route of administration is an oral administration.

In some embodiments, the ATR inhibitor is administered 1 day/week, 2 days/week, or 3 days/week. In some embodiments, the PARP inhibitor is administered 1 day/week, 2 days/week, 3 days/week, or 4 days/week.

In a further aspect, the invention provides a method of inducing cell death in an aberrant cancer cell having a loss of function of ATM, BRCA2, RNAse H2A, RNAse H2B, CDK12, or a combination thereof, or in an ALT+ cancer cell, the method comprising contacting the cell with an effective amount of an ATR inhibitor and an effective amount of a PARP inhibitor, the effective amounts being sufficient to induce cell death in the aberrant cancer cell; where the PARP inhibitor is veliparib (ABT-888), iniparib (BSI-201), 2X-121, CEP-9722, KU-0059436 (AZD2281), PF-01367338, a pharmaceutically acceptable salt thereof, or a combination thereof.

In some embodiments, the loss of function is a loss of function of ATM. In some embodiments, the loss of function is a loss of function of RNAse H2A. In some embodiments, the loss of function is a loss of function of RNAse H2B. In some embodiments, the loss of function is a loss of function of CDK12. In some embodiments, the loss of function is a loss of function of BRCA2. In some embodiments, the cancer is an ALT+ cancer.

In some embodiments, the ATR inhibitor is a compound of formula (I):

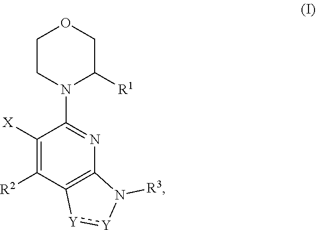

or a pharmaceutically acceptable salt thereof,
where
[symbol] is a double bond, and each Y is independently N or CR⁴; or [symbol] is a single bond, and each Y is independently NR^{Y}, carbonyl, or C(R^{Y})₂; where each R^{Y} is independently H or optionally substituted C₁₋₆ alkyl;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

In some embodiments, the ATR inhibitor is a compound of formula (II):

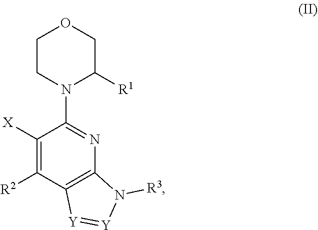

or a pharmaceutically acceptable salt thereof,
where
each Y is independently N or CR⁴;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

In some embodiments, the ATR inhibitor is selected from the group consisting of compounds 43, 57, 62, 87, 93, 94, 95, 99, 100, 106, 107, 108, 109, 111, 112, 113, 114, 115, 116, 118, 119, 120, 121, 122, 123, 135, 147, 148, and pharmaceutically acceptable salts thereof. In some embodiments, the ATR inhibitor is compound 43 or a pharmaceutically acceptable salt thereof. In some embodiments, the ATR inhibitor is compound 121 or a pharmaceutically acceptable salt thereof. In some embodiments, the ATR inhibitor is compound 122 or a pharmaceutically acceptable salt thereof.

### Definitions

The term “aberrant,” as used herein, refers to different from normal. When used to describe enzymatic activity, aberrant refers to activity that is greater or less than a normal control or the average of normal non-diseased control samples. Aberrant activity may refer to an amount of activity that results in a disease, where returning the aberrant activity to a normal or non-disease-associated amount (e.g. by administering a compound or using a method as described herein), results in reduction of the disease or one or more disease symptoms. The aberrant activity can be measured by measuring the modification of a substrate of the enzyme in question; a difference of greater or equal to a 2-fold change in activity could be considered as aberrant. Aberrant activity could also refer to an increased dependence on a particular signaling pathway as a result of a deficiency in a separate complementary pathway

The term “acyl,” as used herein, represents a group —C(═O)—R, where R is alkyl, alkenyl, alkynyl, cycloalkyl, cycloalkenyl, cycloalkynyl, aryl, heteroaryl, or heterocyclyl. Acyl may be optionally substituted as described herein for each respective R group.

The term “adenocarcinoma,” as used herein, represents a malignancy of the arising from the glandular cells that line organs within an organism. Non-limiting examples of adenocarcinomas include non-small cell lung cancer, prostate cancer, pancreatic cancer, esophageal cancer, and colorectal cancer.

The term “alkanoyl,” as used herein, represents a hydrogen or an alkyl group that is attached to the parent molecular group through a carbonyl group and is exemplified by formyl (i.e., a carboxyaldehyde group), acetyl, propionyl, butyryl, and iso-butyryl. Unsubstituted alkanoyl groups contain from 1 to 7 carbons. The alkanoyl group may be unsubstituted of substituted (e.g., optionally substituted C₁₋₇ alkanoyl) as described herein for alkyl group. The ending “-oyl” may be added to another group defined herein, e.g., aryl, cycloalkyl, and heterocyclyl, to define “aryloyl,” “cycloalkanoyl,” and “(heterocyclyl)oyl.” These groups represent a carbonyl group substituted by aryl, cycloalkyl, or heterocyclyl, respectively. Each of “aryloyl,” “cycloalkanoyl,” and “(heterocyclyl)oyl” may be optionally substituted as defined for “aryl,” “cycloalkyl,” or “heterocyclyl,” respectively.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkoxy,” as used herein, represents a chemical substituent of formula-OR, where R is a C₁₋₆ alkyl group, unless otherwise specified. In some embodiments, the alkyl group can be further substituted as defined herein. The term “alkoxy” can be combined with other terms defined herein, e.g., aryl, cycloalkyl, or heterocyclyl, to define an “aryl alkoxy,” “cycloalkyl alkoxy,” and “(heterocyclyl)alkoxy” groups. These groups represent an alkoxy that is substituted by aryl, cycloalkyl, or heterocyclyl, respectively. Each of “aryl alkoxy,” “cycloalkyl alkoxy,” and “(heterocyclyl)alkoxy” may optionally substituted as defined herein for each individual portion.

The term “alkoxyalkyl,” as used herein, represents a chemical substituent of formula -L-O—R, where L is C₁₋₆ alkylene, and R is C₁₋₆ alkyl. An optionally substituted alkoxyalkyl is an alkoxyalkyl that is optionally substituted as described herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: amino; aryl; aryloxy; azido; cycloalkyl; cycloalkoxy; cycloalkenyl; cycloalkynyl; halo; heterocyclyl; (heterocyclyl)oxy; heteroaryl; hydroxy; nitro; thiol; silyl; cyano; alkylsulfonyl; alkylsulfinyl; alkylsulfenyl; ═O; ═S; —SO₂R, where R is amino or cycloalkyl; ═NR′, where R′ is H, alkyl, aryl, or heterocyclyl. Each of the substituents may itself be unsubstituted or, valency permitting, substituted with unsubstituted substituent(s) defined herein for each respective group.

The term “alkylene,” as used herein, refers to a divalent alkyl group. An optionally substituted alkylene is an alkylene that is optionally substituted as described herein for alkyl.

The term “alkylamino,” as used herein, refers to a group having the formula —N(R^{N1})₂ or —NHR^{N1}, in which R^{N1} is alkyl, as defined herein. The alkyl portion of alkylamino can be optionally substituted as defined for alkyl. Each optional substituent on the substituted alkylamino may itself be unsubstituted or, valency permitting, substituted with unsubstituted substituent(s) defined herein for each respective group.

The term “alkylsulfenyl,” as used herein, represents a group of formula —S-(alkyl). Alkylsulfenyl may be optionally substituted as defined for alkyl.

The term “alkylsulfinyl,” as used herein, represents a group of formula —S(O)-(alkyl). Alkylsulfinyl may be optionally substituted as defined for alkyl.

The term “alkylsulfonyl,” as used herein, represents a group of formula —S(O)₂-(alkyl). Alkylsulfonyl may be optionally substituted as defined for alkyl.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. The alkynyl groups may be unsubstituted or substituted (e.g., optionally substituted alkynyl) as defined for alkyl.

The term “ALT+ cancer,” as used herein, refers to cancers utilizing homologous recombination-based pathway called alternative lengthening of telomeres (ALT) to extend and maintain telomeres. ALT+ cells may be identified using techniques known in the art. For example, ALT+ cells exhibit one or more of ALT-associated PML bodies, heterogeneous telomere length, abundant extrachromosomal telomere repeat (ECTR), and high levels of telomere sister chromatid exchange (T-SCE). See Bryan et al., EMBO J, 14:4240-4248, 1995; Dunham et al., Nat Genet., 26:447-450, 2000; Muntoni et al., Hum. Mol. Genet., 18:1017-1027, 2009; Yeager et al., Cancer Res., 59:4175-4179, 1999; and Cesare et al., Mol. Cell. Biol., 247:765-772, 2004. ALT+ cancer (e.g., ALT+ cancer cell) may be an ALT+ mesenchymal cancer (e.g., an ALT+ mesenchymal cancer cell). Non-limiting examples of ALT+ cancers include leiomyosarcoma, liposarcoma, glioblastoma, and neuroendocrine pancreatic cancer.

The term “amino,” as used herein, represents —N(R^{N1})₂, where, if amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —COOR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., NHR^{N1}), where R^{N1} is independently —OH, SO₂OR^{N2}, —SO₂RN², —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In some embodiments, an amino group is —NHR^{N1}, in which R^{N1} is optionally substituted alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “arylene,” as used herein, refers to a divalent aryl group. An optionally substituted arylene is an arylene that is optionally substituted as described herein for aryl.

The term “aryloxy,” as used herein, represents a chemical substituent of formula —OR, where R is an aryl group, unless otherwise specified. In optionally substituted aryloxy, the aryl group is optionally substituted as described herein for aryl.

The term “ATM,” as used herein, represents ATM serine/threonine kinase.

The term “ATR inhibitor,” as used herein, represents a compound that upon contacting the enzyme ATR kinase, whether in vitro, in cell culture, or in an animal, reduces the activity of ATR kinase, such that the measured ATR kinase IC₅₀ is 10 μM or less (e.g., 5 μM or less or 1 μM or less). For certain ATR inhibitors, the ATR kinase IC₅₀ may be 100 nM or less (e.g., 10 nM or less, or 1 nM or less) and could be as low as 100 μM or 10 μM. Preferably, the ATR kinase IC₅₀ is 0.1 nM to 1 μM (e.g., 0.1 nM to 750 nM, 0.1 nM to 500 nM, or 0.1 nM to 250 nM).

The term “ATR kinase,” as used herein, refers to Ataxia-telangiectasia and RAD-3-related protein kinase.

The term “azido,” as used herein, represents an —N₃ group.

The term “BRCA2,” as used herein, represents a breast cancer type 2 susceptibility gene or protein.

The term “cancer,” as used herein, refers to all types of cancer, neoplasm or malignant tumors found in mammals (e.g. humans), including leukemia, carcinomas and sarcomas. Non-limiting examples of cancers that may be treated with a compound or method provided herein include prostate cancer, thyroid cancer, endocrine system cancer, brain cancer, breast cancer, cervix cancer, colon cancer, head & neck cancer, liver cancer, kidney cancer, lung cancer, non-small cell lung cancer, melanoma, mesothelioma, ovarian cancer, sarcoma, stomach cancer, uterus cancer, medulloblastoma, ampullary cancer, colorectal cancer, and pancreatic cancer. Additional non-limiting examples may include, Hodgkin's disease, Non-Hodgkin's lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphoma, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, and prostate cancer.

The term “carbocyclic,” as used herein, represents an optionally substituted C3-16 monocyclic, bicyclic, or tricyclic structure in which the rings, which may be aromatic or non-aromatic, are formed by carbon atoms. Carbocyclic structures include cycloalkyl, cycloalkenyl, cycloalkynyl, and certain aryl groups.

The term “carbonyl,” as used herein, represents a —C(O)— group.

The term “carcinoma,” as used herein, refers to a malignant new growth made up of epithelial cells tending to infiltrate the surrounding tissues and give rise to metastases. Non-limiting examples of carcinomas that may be treated with a compound or method provided herein include, e.g., medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifomi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “cyano,” as used herein, represents —CN group.

The term “cycloalkenyl,” as used herein, refers to a non-aromatic carbocyclic group having at least one double bond in the ring and from three to ten carbons (e.g., a C₃₋₁₀ cycloalkenyl), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenyl alkyl,” as used herein, represents an alkyl group substituted with a cycloalkenyl group, each as defined herein. The cycloalkenyl and alkyl portions may be substituted as the individual groups defined herein.

The term “cycloalkoxy,” as used herein, represents a chemical substituent of formula-OR, where R is cycloalkyl group, unless otherwise specified. In some embodiments, the cycloalkyl group can be further substituted as defined herein.

The term “cycloalkyl,” as used herein, refers to a cyclic alkyl group having from three to ten carbons (e.g., a C_{3-C10} cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; heteroaryl; hydroxy; nitro; thiol; silyl; cyano; ═O; ═S; —SO₂R, where R is amino or cycloalkyl; ═NR′, where R′ is H, alkyl, aryl, or heterocyclyl; or —CON(R^{A})₂, where each R^{A} is independently H or alkyl, or both R^{A}, together with the atom to which they are attached, combine to form heterocyclyl. Each of the substituents may itself be unsubstituted or substituted with unsubstituted substituent(s) defined herein for each respective group.

The term “cycloalkyl alkyl,” as used herein, represents an alkyl group substituted with a cycloalkyl group, each as defined herein. The cycloalkyl and alkyl portions may be optionally substituted as the individual groups described herein.

The term “cycloalkylene,” as used herein, represents a divalent cycloalkyl group. An optionally substituted cycloalkylene is a cycloalkylene that is optionally substituted as described herein for cycloalkyl.

The term “cycloalkynyl,” as used herein, refers to a monovalent carbocyclic group having one or two carbon-carbon triple bonds and having from eight to twelve carbons, unless otherwise specified. Cycloalkynyl may include one transannular bond or bridge. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as defined for cycloalkyl.

“Disease” or “condition” refer to a state of being or health status of a patient or subject capable of being treated with the compounds or methods provided herein.

The term “halo,” as used herein, represents a halogen selected from bromine, chlorine, iodine, and fluorine.

The term “heteroalkyl,” as used herein refers to an alkyl, alkenyl, or alkynyl group interrupted once by one or two heteroatoms; twice, each time, independently, by one or two heteroatoms; three times, each time, independently, by one or two heteroatoms; or four times, each time, independently, by one or two heteroatoms. Each heteroatom is, independently, O, N, or S. In some embodiments, the heteroatom is O or N. None of the heteroalkyl groups includes two contiguous oxygen or sulfur atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteratom. Thus, the substituent bonded to the heteroatom, valency permitting, is selected from the group consisting of ═O, —N(R^{N2})₂, —SO₂OR^{N3}, —SO₂RN², —SOR^{N3}, —COOR^{N3}, an N protecting group, alkyl, alkenyl, alkynyl, aryl, cycloalkyl, cycloalkenyl, cycloalkynyl, heterocyclyl, or cyano, where each R^{N2} is independently H, alkyl, cycloalkyl, cycloalkenyl, cycloalkynyl, aryl, or heterocyclyl, and each R^{N3} is independently alkyl, cycloalkyl, cycloalkenyl, cycloalkynyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group.

The term “heteroaryl alkyl,” as used herein, represents an alkyl group substituted with a heteroaryl group, each as defined herein. The heteroaryl and alkyl portions may be optionally substituted as the individual groups described herein.

The term “heteroarylene,” as used herein, represents a divalent heteroaryl. An optionally substituted heteroarylene is a heteroarylene that is optionally substituted as described herein for heteroaryl.

The term “heteroaryloxy,” as used herein, refers to a structure —OR, in which R is heteroaryl. Heteroaryloxy can be optionally substituted as defined for heterocyclyl.

The term “heterocyclyl,” as used herein, represents a monocyclic, bicyclic, tricyclic, or tetracyclic ring system having fused, bridging, and/or spiro 3-, 4-, 5-, 6-, 7-, or 8-membered rings, unless otherwise specified, containing one, two, three, or four heteroatoms independently selected from the group consisting of nitrogen, oxygen, and sulfur. In some embodiments, “heterocyclyl” is a monocyclic, bicyclic, tricyclic, or tetracyclic ring system having fused or bridging 5-, 6-, 7-, or 8-membered rings, unless otherwise specified, containing one, two, three, or four heteroatoms independently selected from the group consisting of nitrogen, oxygen, and sulfur. Heterocyclyl can be aromatic or non-aromatic. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups include from 1 to 16 carbon atoms unless otherwise specified. Certain heterocyclyl groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four, five, or six substituents independently selected from the group consisting of alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “heterocyclyl alkyl,” as used herein, represents an alkyl group substituted with a heterocyclyl group, each as defined herein. The heterocyclyl and alkyl portions may be optionally substituted as the individual groups described herein.

The term “heterocyclylene,” as used herein, represents a divalent heterocyclyl. An optionally substituted heterocyclylene is a heterocyclylene that is optionally substituted as described herein for heterocyclyl.

The term “(heterocyclyl)oxy,” as used herein, represents a chemical substituent of formula —OR, where R is a heterocyclyl group, unless otherwise specified. (Heterocyclyl)oxy can be optionally substituted in a manner described for heterocyclyl.

The terms “hydroxyl” and “hydroxy,” as used interchangeably herein, represent an —OH group.

The term “isotopically enriched,” as used herein, refers to the pharmaceutically active agent with the isotopic content for one isotope at a predetermined position within a molecule that is at least 100 times greater than the natural abundance of this isotope. For example, a composition that is isotopically enriched for deuterium includes an active agent with at least one hydrogen atom position having at least 100 times greater abundance of deuterium than the natural abundance of deuterium. Preferably, an isotopic enrichment for deuterium is at least 1000 times greater than the natural abundance of deuterium. More preferably, an isotopic enrichment for deuterium is at least 4000 times greater (e.g., at least 4750 times greater, e.g., up to 5000 times greater) than the natural abundance of deuterium.

The term “leukemia,” as used herein, refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, e.g., acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “lymphoma,” as used herein, refers to a cancer arising from cells of immune origin. Non-limiting examples of T and B cell lymphomas include non-Hodgkin lymphoma and Hodgkin disease, diffuse large B-cell lymphoma, follicular lymphoma, mucosa-associated lymphatic tissue (MALT) lymphoma, small cell lymphocytic lymphoma-chronic lymphocytic leukemia, Mantle cell lymphoma, mediastinal (thymic) large B-cell lymphoma, lymphoplasmacytic lymphoma-Waldenstrom macroglobulinemia, peripheral T-cell lymphoma (PTCL), angioimmunoblastic T-cell lymphoma (AITL)/follicular T-cell lymphoma (FTCL), anaplastic large cell lymphoma (ALCL), enteropathy-associated T-cell lymphoma (EATL), adult T-cell leukaemia/lymphoma (ATLL), or extranodal NK/T-cell lymphoma, nasal type.

The term “melanoma,” as used herein, is taken to mean a tumor arising from the melanocytic system of the skin and other organs. Melanomas that may be treated with a compound or method provided herein include, e.g., acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

The term “nitro,” as used herein, represents an —NO₂ group.

The term “oxo,” as used herein, represents a divalent oxygen atom (e.g., the structure of oxo may be shown as ═O).

The term “PARP inhibitor,” as used herein and unless specified otherwise, represents a compound of formula (III):

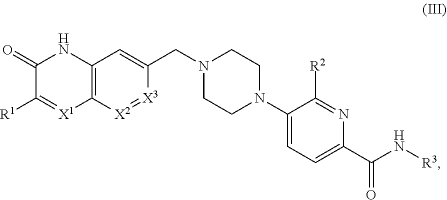

where
X¹ and X² are each independently selected from N and C(H),
X³ is independently selected from N and C(R⁴), where R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹ is C(H), and X³ is C(R⁴), and
when X³ is N, then X¹ and X² are both C(H).

The term “PARP,” as used herein, refers to poly ADP ribose polymerase (PARP).

The term “Ph,” as used herein, represents phenyl.

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable excipient” or “pharmaceutically acceptable carrier,” as used interchangeably herein, refers to any ingredient other than the compounds described herein (e.g., a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quatemary ammonium, and amine cations, including, but not limited to ammonium, tetramethylammonium, tetraethylammonium, methylamine, dimethylamine, trimethylamine, triethylamine, ethylamine, and the like.

The term “protecting group,” as used herein, represents a group intended to protect a hydroxy, an amino, or a carbonyl from participating in one or more undesirable reactions during chemical synthesis. The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino, amido, heterocyclic N—H, or hydrazine) group from participating in one or more undesirable reactions during chemical synthesis. Commonly used O- and N-protecting groups are disclosed in Greene, “Protective Groups in Organic Synthesis,” 3rd Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, a-chlorobutyryl, benzoyl, 4-chlorobenzoyl, 4-bromobenzoyl, t-butyldimethylsilyl, tri-iso-propylsilyloxymethyl, 4,4-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2, -trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p methoxybenzyloxycarbonyl, p-nitrobenzyloxycarbonyl, 2-nitrobenzyloxycarbonyl, p bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, a,a-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2, -trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, p-methoxybenzyl, 2,4-dimethoxybenzyl, triphenylmethyl, benzyloxymethyl, and the like, silylalkylacetal groups such as [2-(trimethylsilyl)ethoxy]methyl and silyl groups such as trimethylsilyl, and the like. Useful N-protecting groups are formyl, acetyl, benzoyl, pivaloyl, t-butylacetyl, alanyl, phenylsulfonyl, benzyl, dimethoxybenzyl, [2-(trimethylsilyl)ethoxy]methyl (SEM), tetrahydropyranyl (THP), t-butyloxycarbonyl (Boc), and benzyloxycarbonyl (Cbz).

The term “RNAse H2A,” as used herein, refers to Ribonuclease H2, subunit A.

The term “RNAse H2B,” as used herein, refers to Ribonuclease H2, subunit B.

The term “sarcoma” generally refers to a tumor which is made up of a substance like the embryonic connective tissue and is generally composed of closely packed cells embedded in a fibrillar or homogeneous substance. Non-limiting examples of sarcomas that may be treated with a compound or method provided herein include, e.g., a chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “tautomer” refers to structural isomers that readily interconvert, often by relocation of a proton. Tautomers are distinct chemical species that can be identified by differing spectroscopic characteristics, but generally cannot be isolated individually. Non-limiting examples of tautomers include ketone—enol, enamine—imine, amide—imidic acid, nitroso—oxime, ketene—ynol, and amino acid—ammonium carboxylate.

The term “therapeutically effective amount,” as used herein, means the amount of a compound or a pharmaceutically acceptable salt thereof that, in a combination of an ATR inhibitor and PARP inhibitor, is sufficient to treat cancer. Typically, a therapeutically effective amount is a subtherapeutic regimen.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer.

The term “subtherapeutic regimen,” as used herein, refers to a dosing regimen that is at least 5% less (e.g., at least 10%, 20%, 50%, 80%, 90%, or even 95%) than the lowest standard recommended dosing regimen of a particular compound formulated for a given route of administration for treatment of cancer. A subtherapeutic regimen of a compound may be therapeutically ineffective for the compound in a monotherapy regimen. In the methods of the invention, a therapeutically effective amount of a PARP inhibitor is preferably a subtherapeutic regimen (e.g., a regimen that is therapeutically ineffective for the PARP inhibitor in a monotherapy regimen). A subtherapeutic regimen of a PARP inhibitor that is formulated for oral administration may differ from a subtherapeutic regimen of the same agent formulated for intratumoral administration. A subtherapeutic regimen may include a “subtherapeutic starting regimen” and a “subtherapeutic maintenance regimen.” A “subtherapeutic starting regimen” of a compound (e.g., a PARP inhibitor) is lower than the lowest standard starting dosage of the same compound (e.g., a PARP inhibitor). Similarly, a “subtherapeutic maintenance regimen” of a compound (e.g., a PARP inhibitor) is lower than the lowest standard maintenance regimen of the same compound (e.g., a PARP inhibitor). Typically, the subtherapeutic regimen is at least 1% of the lowest standard subtherapeutic regimen.

“Treatment” and “treating,” as used herein, refer to the medical management of a subject with the intent to improve, ameliorate, stabilize, prevent or cure a disease or condition. This term includes active treatment (treatment directed to improve the disease or condition); causal treatment (treatment directed to the cause of the associated disease or condition); palliative treatment (treatment designed for the relief of symptoms of the disease or condition); preventative treatment (treatment directed to minimizing or partially or completely inhibiting the development of the associated disease or condition); and supportive treatment (treatment employed to supplement another therapy). A disease or condition may be a cancer. Non-limiting examples of cancers include, e.g., renal cell carcinoma, mature B-cell neoplasms, endometrial cancer, ovarian cancer, colorectal cancer, skin cancer (non-melanoma), small bowel cancer, non-small cell lung cancer, melanoma, bladder cancer, pancreatic cancer, head and neck cancer, mesothelioma, glioma, prostate cancer, breast cancer, and esophagogastric cancer.

If any publication incorporated herein by reference contains a definition not consistent with a definition presented herein, the latter definition prevails.

### DETAILED DESCRIPTION

In general, the invention relates to a combination of an ATR inhibitor, or a pharmaceutically acceptable salt thereof, and a PARP inhibitor, or a pharmaceutically acceptable salt thereof for the treatment of cancers or for inducing cell death in cancer cells. The cancers included herein may be, e.g., cancers having a loss of function of ATM, BRCA2, RNAse H2A, RNAse H2B, CDK12, or a combination thereof. Alternatively, the cancer may be, e.g., an ALT+ cancer.

Advantageously, an ATR inhibitor and a PARP inhibitor act synergistically to induce cell death in cancer cells having a loss of function of ATM, BRCA2, RNAse H2A, RNAse H2B, or CDK12, or in ALT+ cancer cells. Advantageously, combination cancer therapies including an ATR inhibitor and a PARP inhibitor may exhibit reduced morbidities, as ATR inhibitor and PARP inhibitor dosages may be reduced, e.g., relative to those administered in corresponding monotherapies. Thus, ATR and PARP inhibitors may be used in subtherapeutic regimens in the methods of the invention.

Synergy between ATR inhibitors and PARP inhibitors has been observed, but continuous combination treatment has been historically poorly tolerated in preclinical models (Fang et al., Cancer Cell, 35:851-867, 2019) and there are potentially overlapping toxicities observed for each drug class in the clinic (Sachdev et al., Targeted Oncology, 14:657-679, 2019, Mei et al., J. Hematol. Oncol., 12:43, 2019). Until the present invention, specific populations of cancer patients that could benefit from a combination of an ATR inhibitor and a PARP inhibitor were limited (Kim et al., Clin. Cancer. Res., 23:3097-3108, 2017), especially those that could benefit from the dose reductions for the ATR inhibitors and/or PARP inhibitors.

ATR Inhibitors

ATR inhibitors a compound that upon contacting the enzyme ATR kinase, whether in vitro, in cell culture, or in an animal, reduces the activity of ATR kinase, such that the measured ATR kinase IC₅₀ is 10 μM or less (e.g., 5 μM or less or 1 μM or less). For certain ATR inhibitors, the ATR kinase IC₅₀ may be 100 nM or less (e.g., 10 nM or less, or 1 nM or less) and could be as low as 100 μM or 10 μM. Preferably, the ATR kinase IC₅₀ is 0.1 nM to 1 μM (e.g., 0.1 nM to 750 nM, 0.1 nM to 500 nM, or 0.1 nM to 250 nM).

Non-limiting examples of ATR inhibitors include, e.g.:

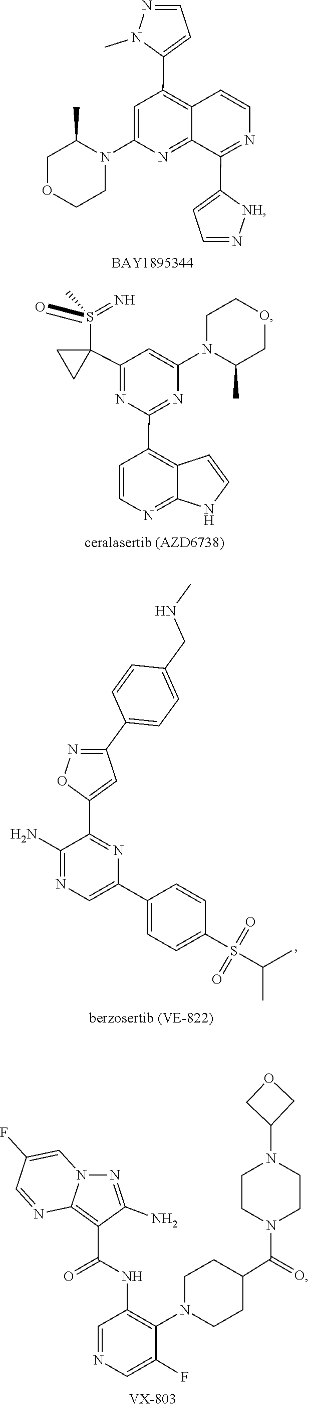

and pharmaceutically acceptable salts thereof.

Non-limiting examples of ATR inhibitors include, e.g., those described in, e.g., International Application Nos. PCT/US2019/051539 and PCT/US2018/034729, each of which is incorporated by reference herein; U.S. Pat. Nos. 9,663,535, 9,549,932, 8,552,004, and 8,841,308, each of which is incorporated by reference herein; and U.S. Patent Application Publication No. 2019/0055240, which is incorporated by reference herein.

In one embodiment. an ATR inhibitor is a compound of formula (I):

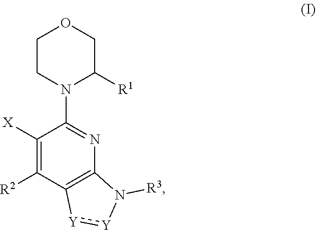

or a pharmaceutically acceptable salt thereof,
where
[symbol] is a double bond, and each Y is independently N or CR⁴; or [symbol] is a single bond, and each Y is independently NR^{Y}, carbonyl, or C(R^{Y})₂; where each R^{Y} is independently H or optionally substituted C₁₋₆ alkyl;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

The ATR inhibitor may be, e.g., a compound of formula (II):

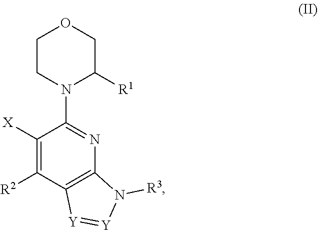

or a pharmaceutically acceptable salt thereof,
where
each Y is independently N or CR⁴;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

In some embodiments, in the compound of formula (II), (I), or (I-b):
each Y is independently N or CR⁴;
R¹ is H or optionally substituted C₁₋₆ alkyl;
R² is optionally substituted C₁₋₆ alkyl, optionally substituted C₃₋₈ cycloalkyl, optionally substituted C₂₋₉ heterocyclyl, optionally substituted C₆₋₁₀ aryl, optionally substituted C₁₋₉ heteroaryl, optionally substituted C₁₋₉ heteroaryl C₁₋₆ alkyl, —N(R⁵)₂, —CON(R⁶)₂, —SO₂N(R⁶)₂, or —SO₂R^{5A};
R³ is optionally substituted C₁₋₉ heteroaryl;
each R⁴ is independently H or optionally substituted C₁₋₆ alkyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}, where each R^{5A} is independently optionally substituted C₁₋₆ alkyl or optionally substituted C₃₋₈ cycloalkyl; or both R⁵, together with the atom to which they are attached, combine to form an optionally substituted C₂₋₉ heterocyclyl;
each R^{5A} is independently optionally substituted C₁₋₆ alkyl or optionally substituted C₃₋₈ cycloalkyl; and
each R⁶ is independently hydrogen, optionally substituted C₁₋₆ alkyl, optionally substituted C₆₋₁₀ aryl C₁₋₆ alkyl, optionally substituted C₆₋₁₀ aryl, or optionally substituted C₁₋₉ heteroaryl; or both R⁶, together with the atom to which they are attached, combine to form an optionally substituted C₂₋₉ heterocyclyl.

Methods of making compounds of formula (I) are described, e.g., in International Application No. PCT/US2019/051539, hereby incorporated by reference.

The ATR inhibitor may be, e.g., a compound of formula (I-a):

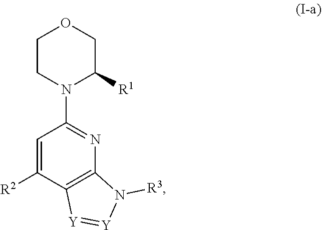

or a pharmaceutically acceptable salt thereof, where Y, R¹, R², R³, and R⁴ are as described for formula (I).

The ATR inhibitor may be, e.g., a compound of formula (I-b):

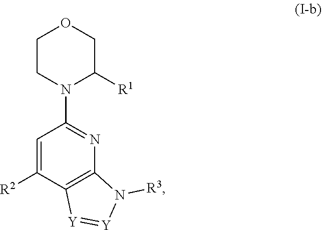

or a pharmaceutically acceptable salt thereof, where Y, R¹, R², R³, and R⁴ are as described for formula (I).

The ATR inhibitor may be, e.g., a compound of formula (IA):

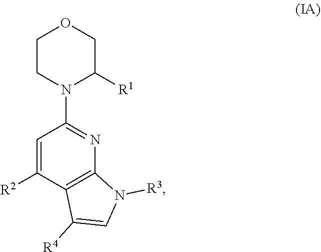

or a pharmaceutically acceptable salt thereof, where R¹, R², R³, and R⁴ are as described for formula (I).

The ATR inhibitor may be, e.g., a compound of formula (IA-a):

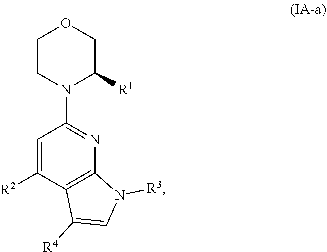

or a pharmaceutically acceptable salt thereof, where R¹, R², R³, and R⁴ are as described for formula (I).

The ATR inhibitor may be, e.g., a compound of Formula (IB):

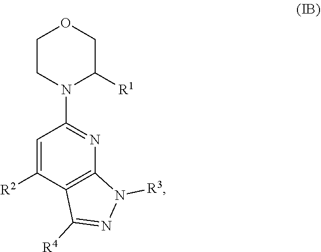

or a pharmaceutically acceptable salt thereof, where R¹, R², R³, and R⁴ are as described for formula (I).

The ATR inhibitor may be, e.g., a compound of formula (IB-a):

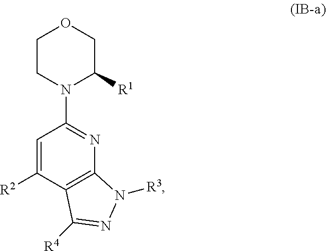

or a pharmaceutically acceptable salt thereof, where R¹, R², R³, and R⁴ are as described for formula (I).

The ATR inhibitor may be, e.g., a compound of Formula (IC):

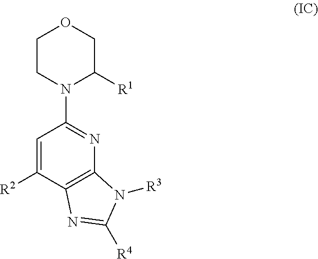

or a pharmaceutically acceptable salt thereof, where R¹, R², R³, and R⁴ are as described for formula (I).

The ATR inhibitor may be, e.g., a compound of formula (IC-a):

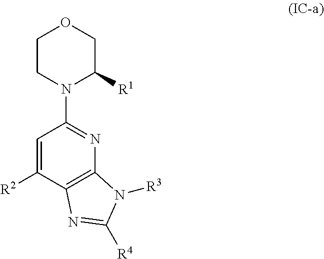

or a pharmaceutically acceptable salt thereof, where R¹, R², R³, and R⁴ are as described for formula (I).

The ATR inhibitor may be, e.g., a compound of formula (ID):

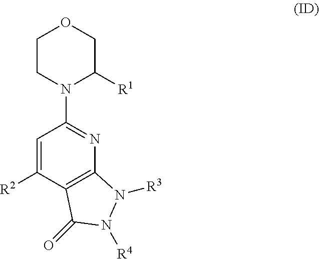

or a pharmaceutically acceptable salt thereof, where R¹, R², R³, and R⁴ are as described for formula (I).

The ATR inhibitor may be, e.g., a compound of formula (ID-a):

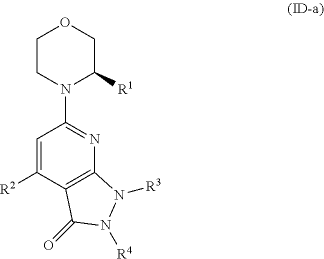

or a pharmaceutically acceptable salt thereof, where R¹, R², R³, and R⁴ are as described for formula (I).

Preferably, R¹ is methyl.

In some embodiments, R² may be, e.g., optionally substituted C₃₋₈ cycloalkyl. For example, R² may be a group of formula (A):

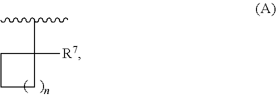

where
n is 0, 1, 2, or 3; and
R⁷ is hydrogen, alkylsulfonyl, cyano, —CON(R^{A})₂, —SON(R^{A})₂, optionally substituted C₁₋₉ heteroaryl, hydroxy, or alkoxy, where each R^{A} is independently H or alkyl; or both R^{A}, together with the atom to which they are attached, combine to form C₂₋₉ heterocyclyl.

In some embodiments, R² may be, e.g., optionally substituted C₁₋₆ alkyl (e.g., optionally substituted tertiary C₃₋₆ alkyl. For example, R² may be a group of formula (B):

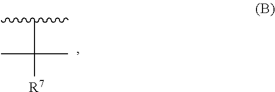

where R⁷ is hydrogen, alkylsulfonyl, cyano, —CON(R^{A})₂, —SON(R^{A})₂, optionally substituted C₁₋₉ heteroaryl, hydroxy, or alkoxy, where each R^{A} is independently H or alkyl; or both R^{A}, together with the atom to which they are attached, combine to form C₂₋₉ heterocyclyl.

In some embodiments, R² may be, e.g., optionally substituted non-aromatic C₂₋₉ heterocyclyl.

In some embodiments, R² may be, e.g.:

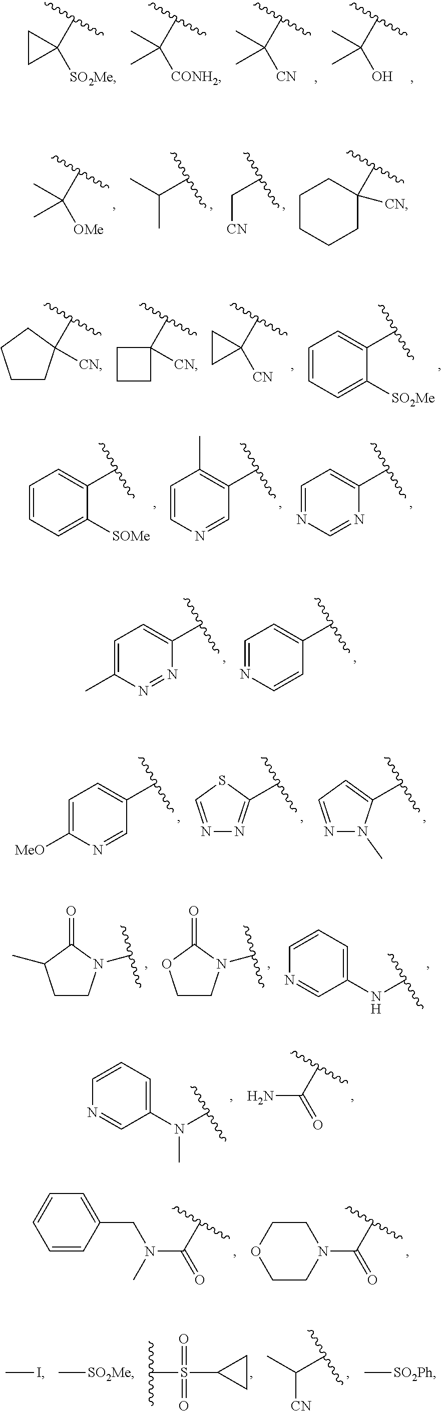

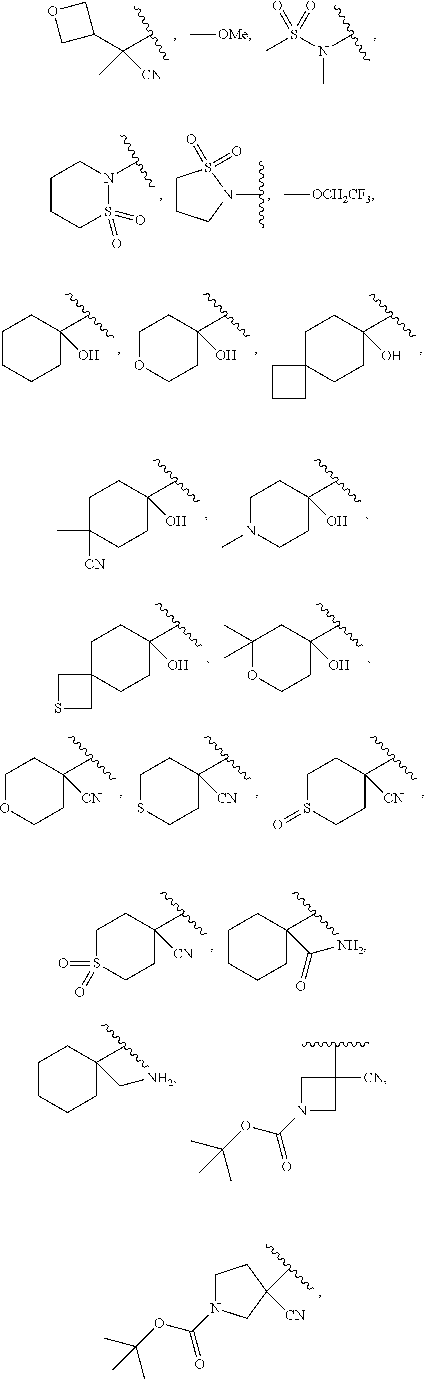

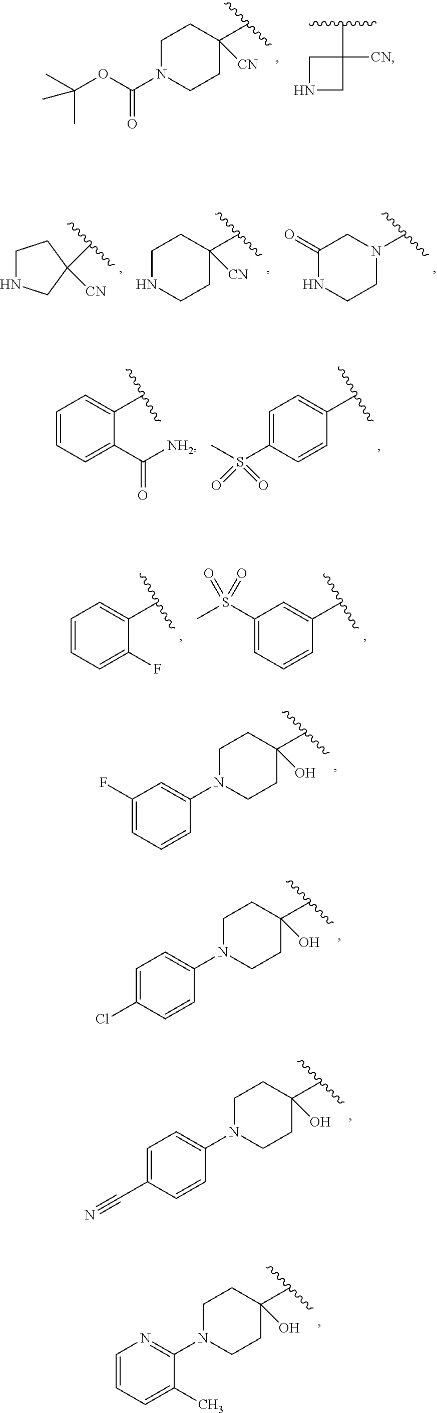

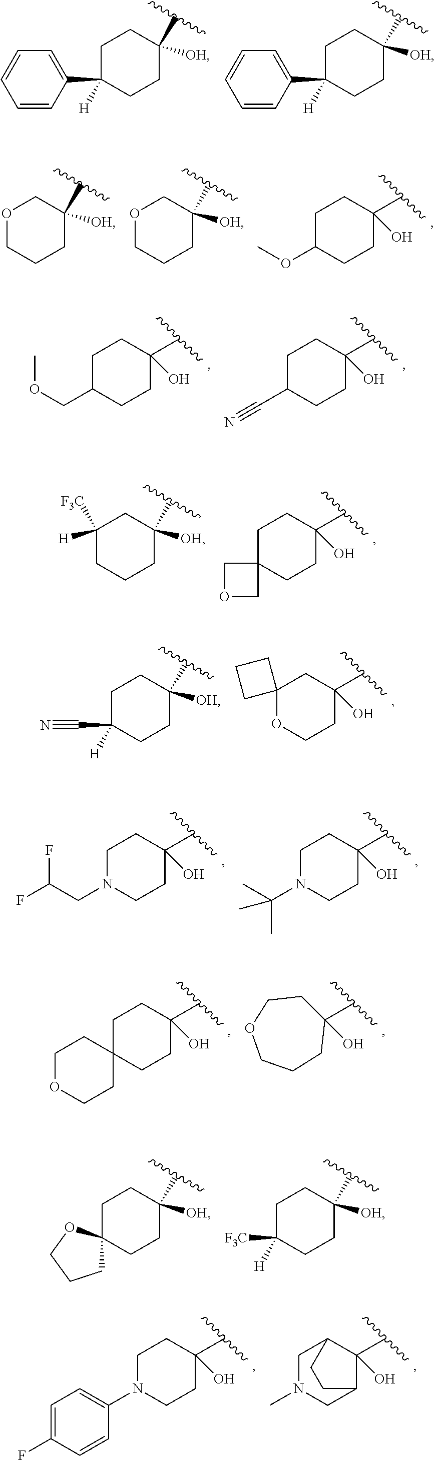

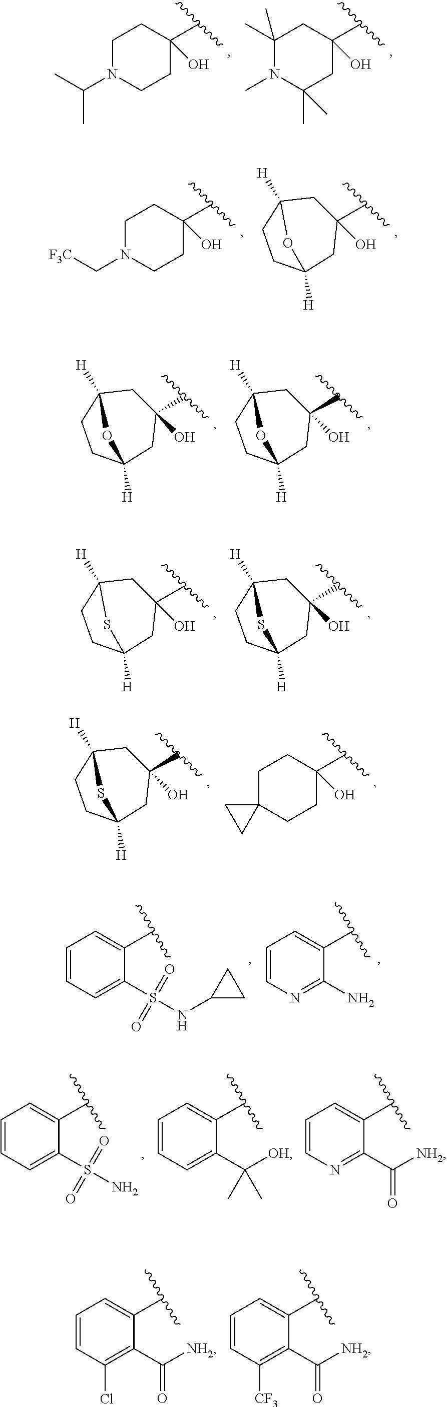

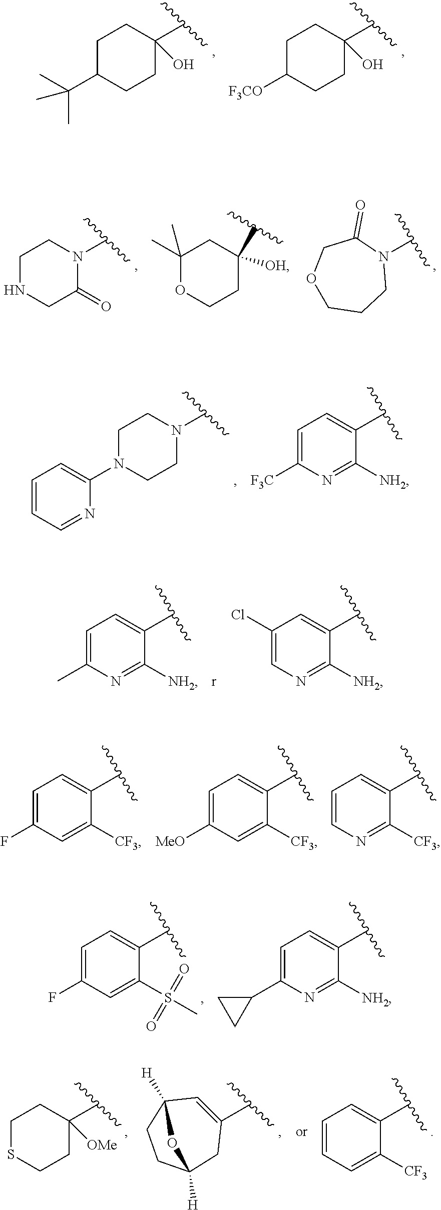

In some embodiments, R³ may be, e.g., optionally substituted, monocyclic C₁₋₉ heteroaryl including at least one nitrogen atom (e.g., two nitrogen atoms). For example, R³ may be a group of formula (C):

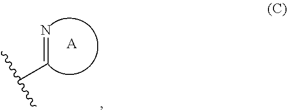

where A is optionally substituted, monocyclic C₁₋₉ heteroaryl ring.

In some embodiments, A may be, e.g., a group of formula (C1):

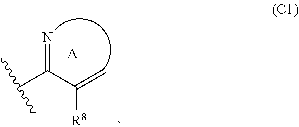

where R⁸ is hydrogen, halogen, or optionally substituted C₁₋₆ alkyl.

In some embodiments, R³ may be, e.g.:

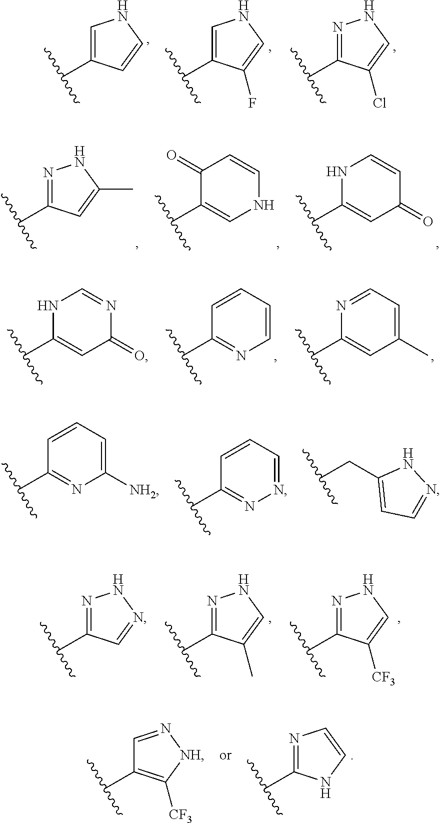

In some embodiments, R³ may be, e.g.:

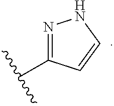 [SMILES: *c1cc[nH]n1]

In some embodiments, R⁴ may be, e.g., hydrogen.

The ATR inhibitor may be, e.g., a compound listed in Table 1 below or a pharmaceutically acceptable salt thereof.

TABLE 1

| | |
|---|---|
| 1 | 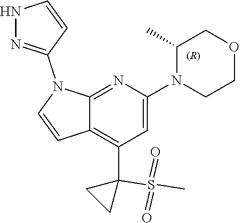 |
| 2 | 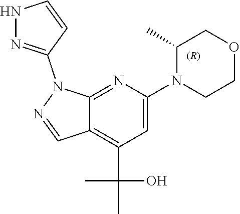 |
| 3 | 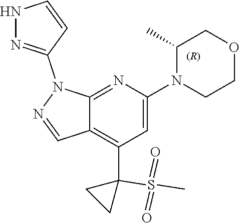 |
| 4 | 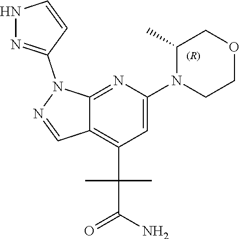 |
| 5 | 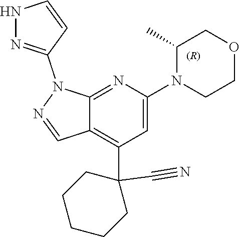 |
| 6 | 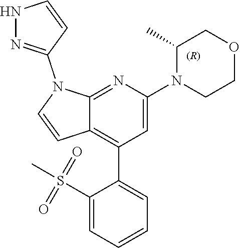 |
| 7 | 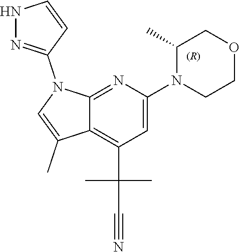 |
| 8 | 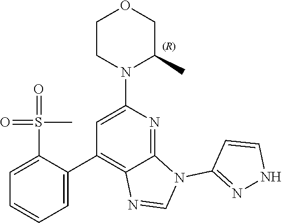 |
| 9 | 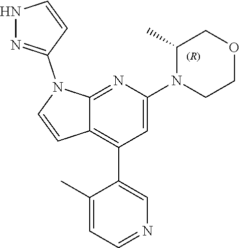 |
| 10 | 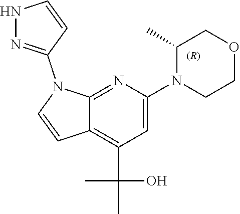 |
| 11 | 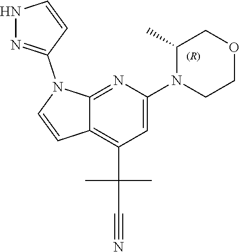 |
| 12 | 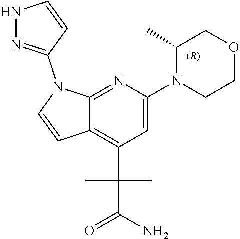 |
| 13 | 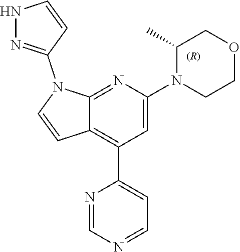 |
| 14 | 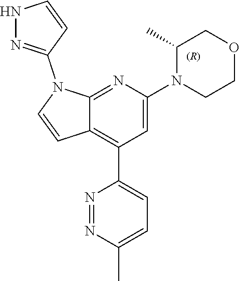 |
| 15 | 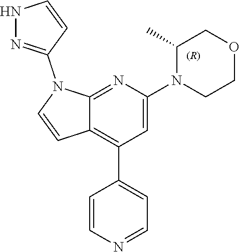 |
| 16 | 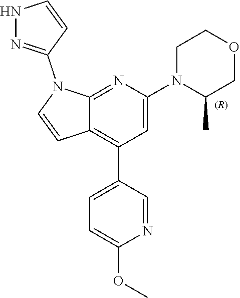 |
| 17 | 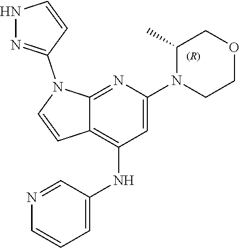 |
| 18 | 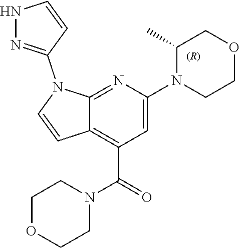 |
| 19 | 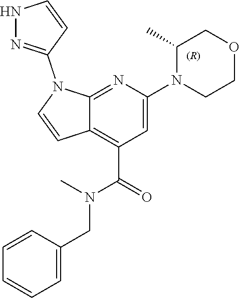 |
| 20 | 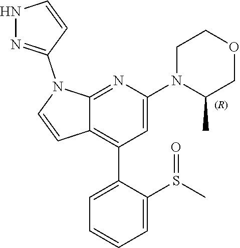 |
| 21 | 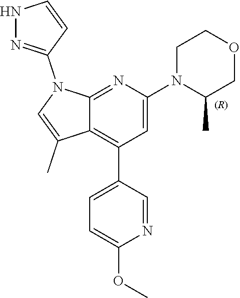 |
| 22 | 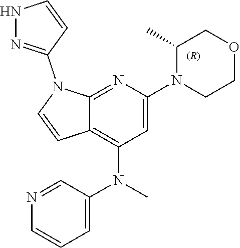 |
| 23 | 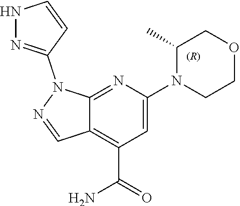 |
| 24 | 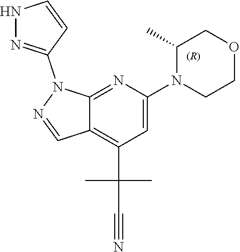 |
| 25 | 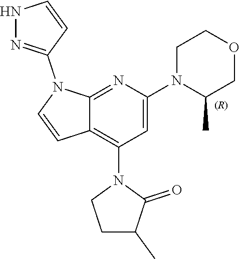 |
| 26 | 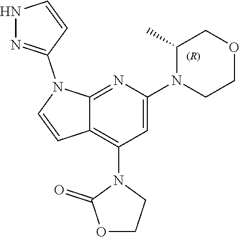 |
| 27 | 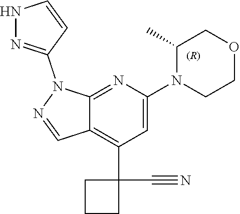 |
| 28 | 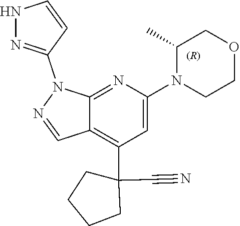 |
| 29 | 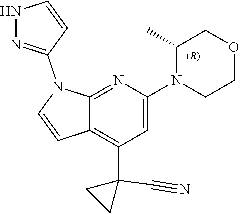 |
| 30 | 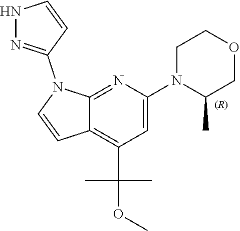 |
| 31 | 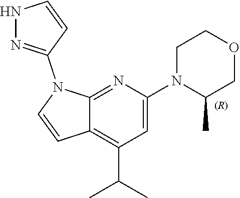 |
| 32 | 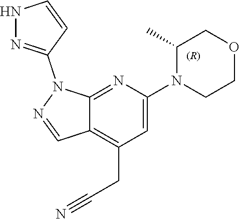 |
| 33 | 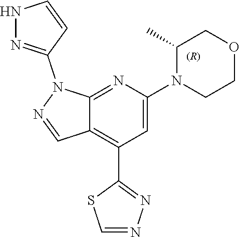 |
| 34 | 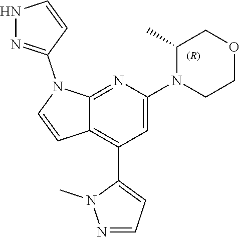 |
| 42 | 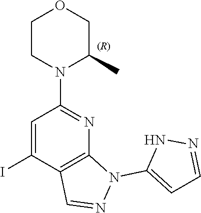 |
| 43 | 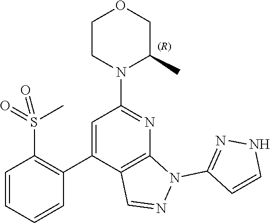 |
| 44 | 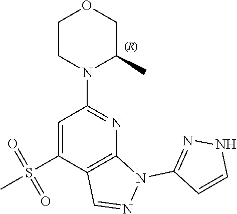 |
| 45 | 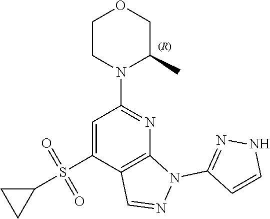 |
| 47 | 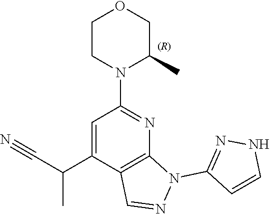 |
| 48 | 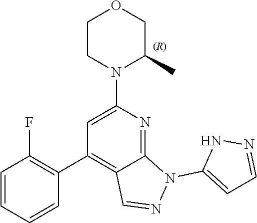 |
| 49 | 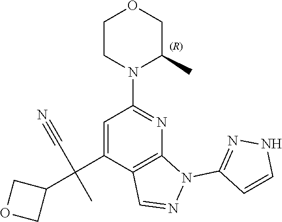 |
| 50 | 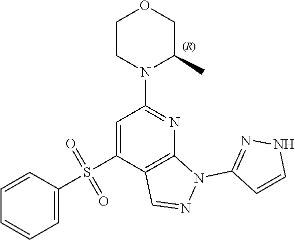 |
| 51 | 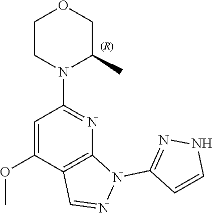 |
| 52 | 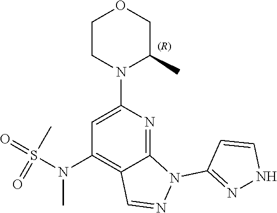 |
| 53 | 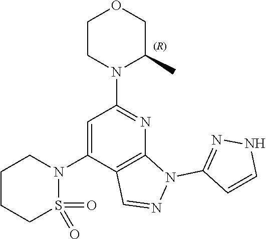 |
| 54 | 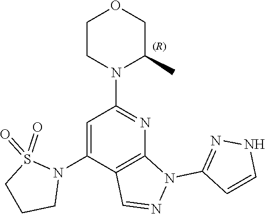 |
| 55 | 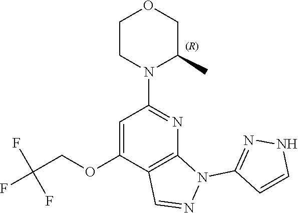 |
| 56 | 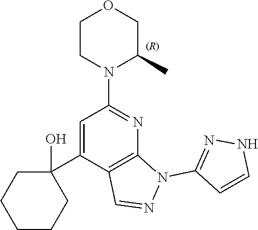 |
| 57 | 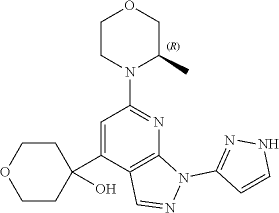 |
| 58 | 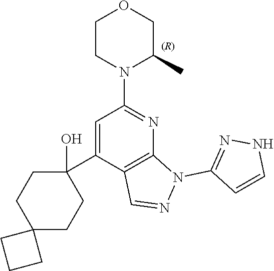 |
| 59 | 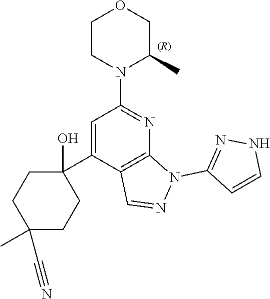 |
| 60 | 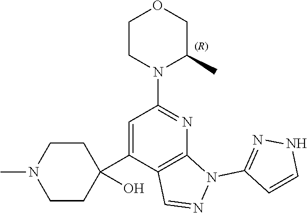 |
| 61 | 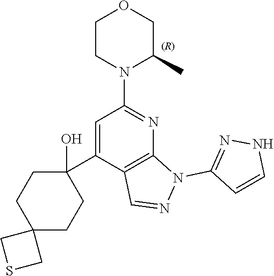 |
| 62 | 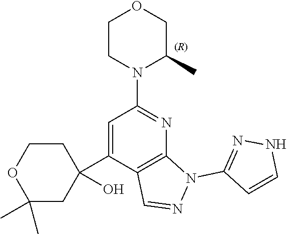 |
| 63 | 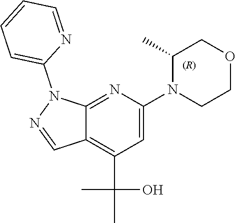 |
| 64 | 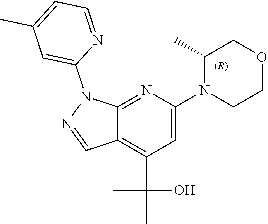 |
| 65 | 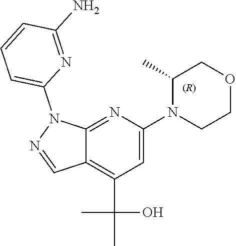 |
| 66 | 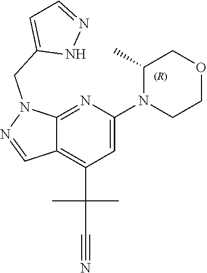 |
| 67 | 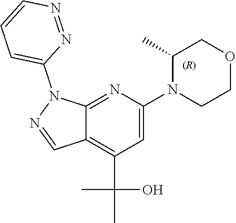 |
| 68 | 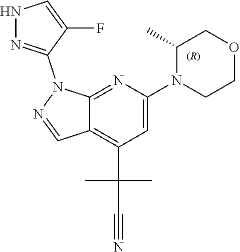 |
| 69 | 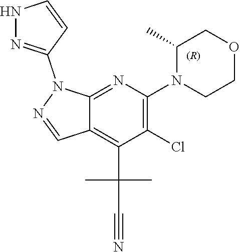 |
| 70 | 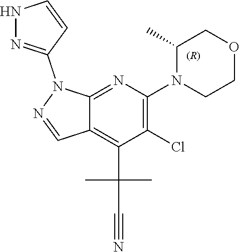 |
| 71 | 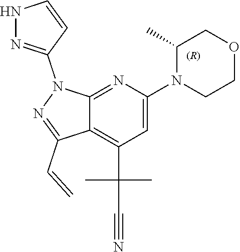 |
| 72 | 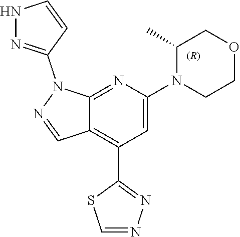 |
| 73 | 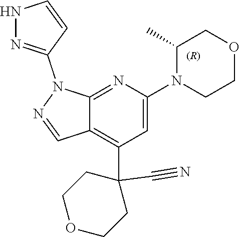 |
| 74 | 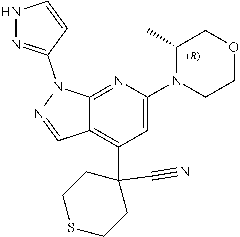 |
| 75 | 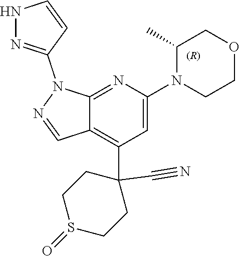 |
| 76 | 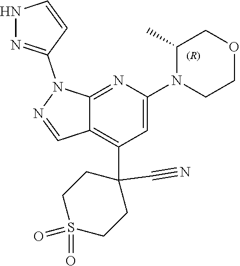 |
| 77 | 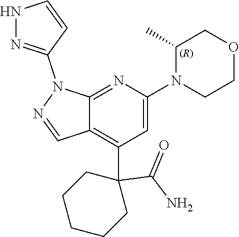 |
| 78 | 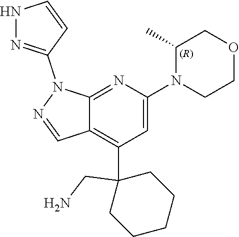 |
| 79 | 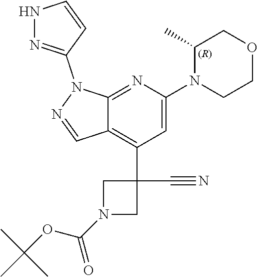 |
| 80 | 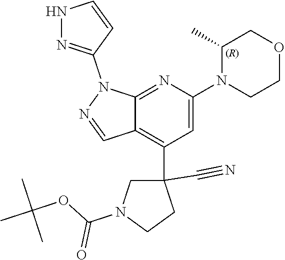 |
| 81 | 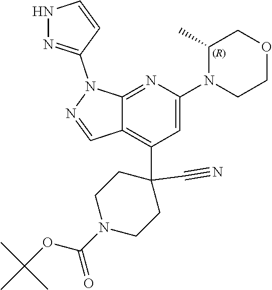 |
| 82 | 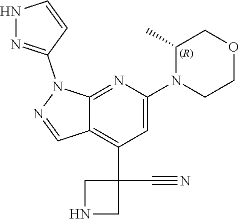 |
| 83 | 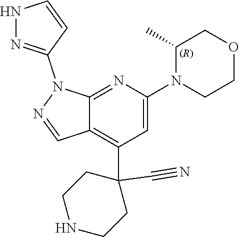 |
| 84 | 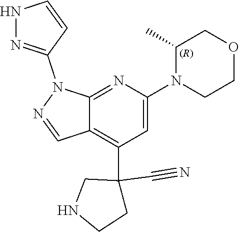 |
| 86 | 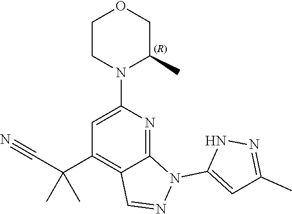 |
| 87 | 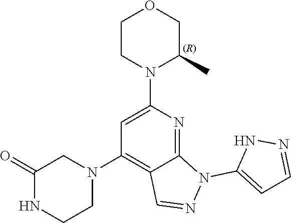 |
| 88 | 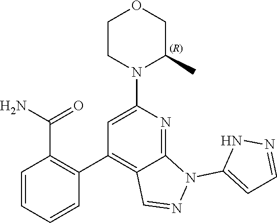 |
| 89 | 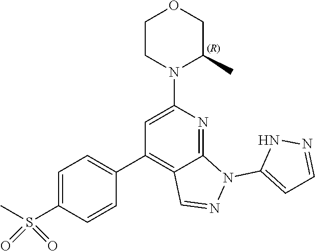 |
| 90 | 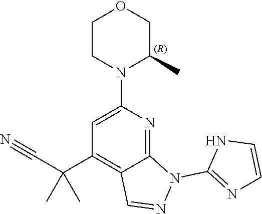 |
| 91 | 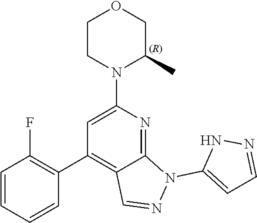 |
| 92 | 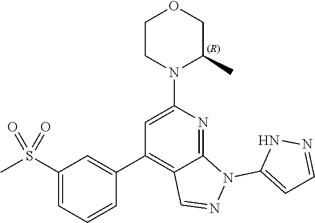 |
| 93 | 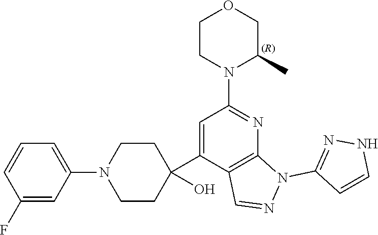 |
| 94 | 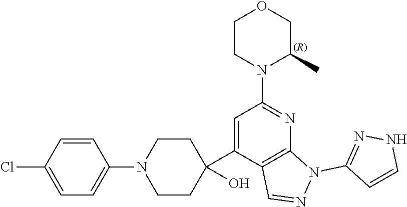 |
| 95 | 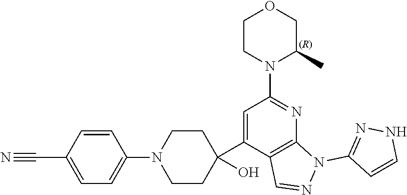 |
| 96 | 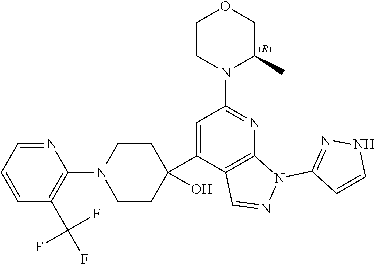 |
| 97 | 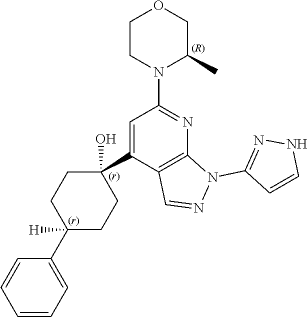 |
| 98 | 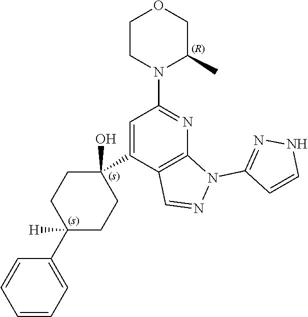 |
| 99 | 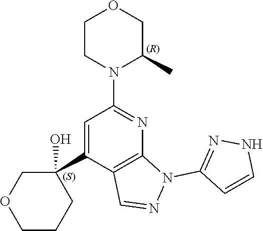 |
| 100 | 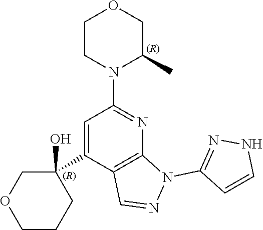 |
| 101 | 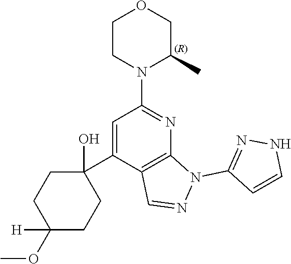 |
| 102 | 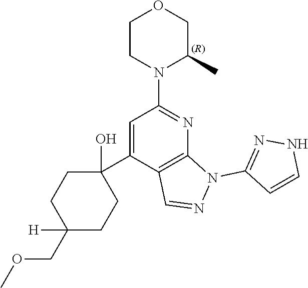 |
| 103 | 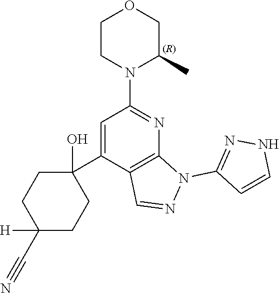 |
| 104 | 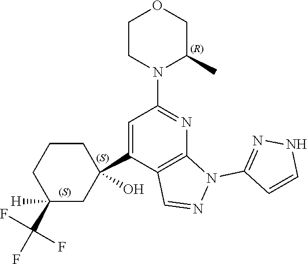 |
| 105 | 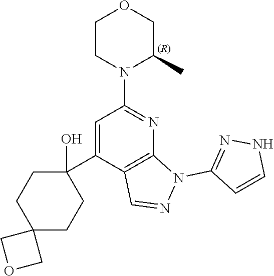 |
| 106 | 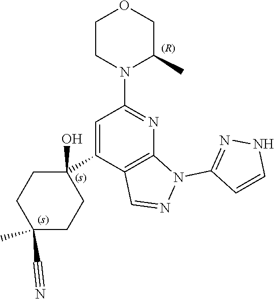 |
| 107 | 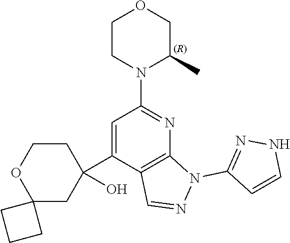 |
| 108 | 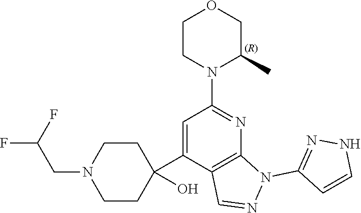 |
| 109 | 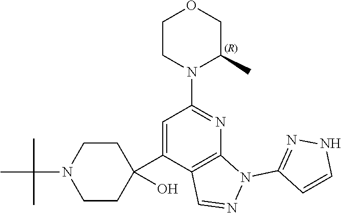 |
| 110 | 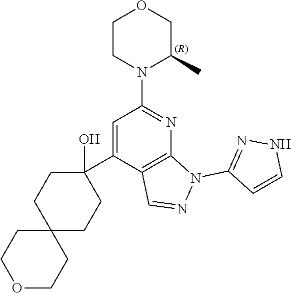 |
| 111 | 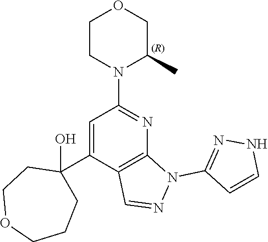 |
| 112 | 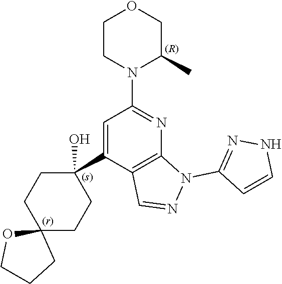 |
| 113 | 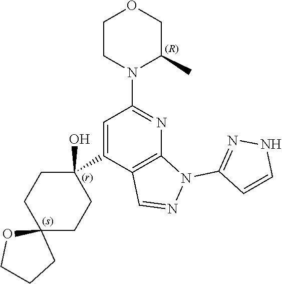 |
| 114 | 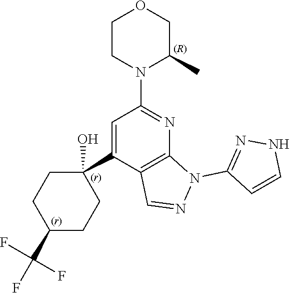 |
| 115 | 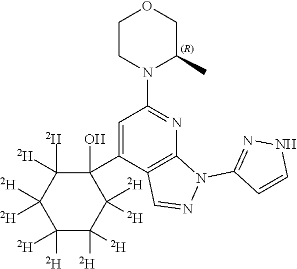 |
| 116 | 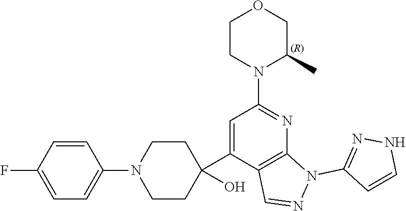 |
| 117 | 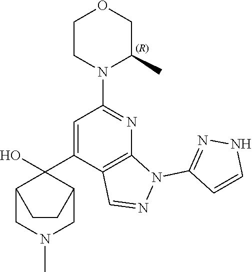 |
| 118 | 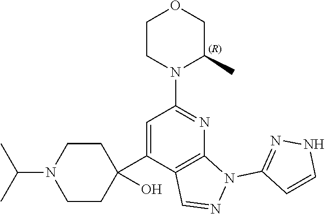 |
| 119 | 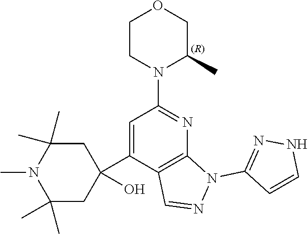 |
| 120 | 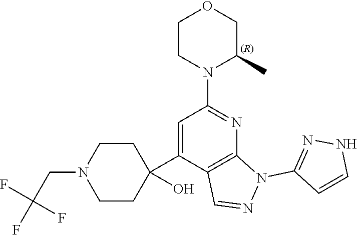 |
| 121 | 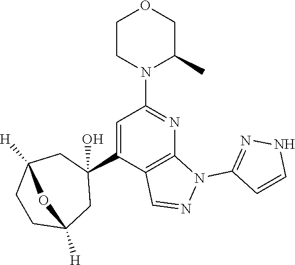 [SMILES: C[C@@H]1COCCN1c1cc([C@@]2(O)C[C@H]3CC[C@@H](C2)O3)c2cnn(-c3cc[nH]n3)c2n1] |
| 122 | 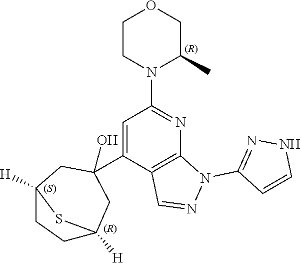 |
| 123 | 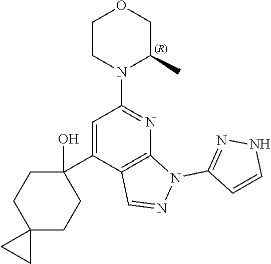 |
| 124 | 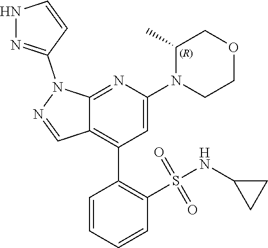 |
| 125 | 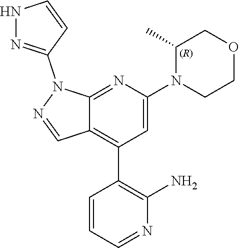 |
| 126 | 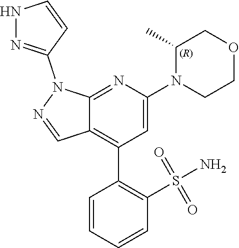 |
| 127 | 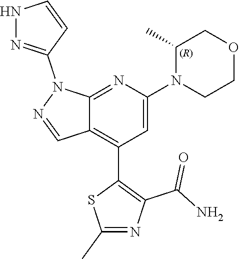 |
| 128 | 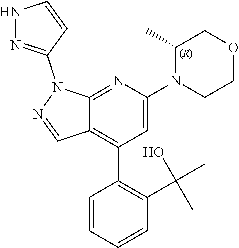 |
| 129 | 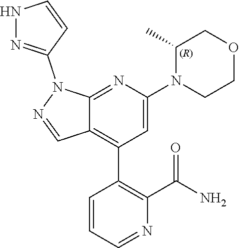 |
| 130 | 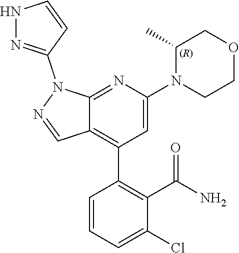 |
| 131 | 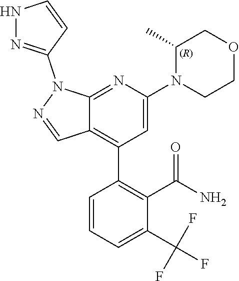 |
| 132 | 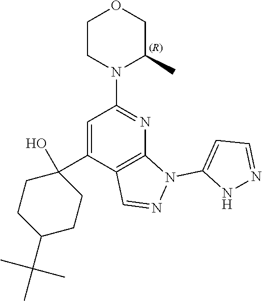 |
| 133 | 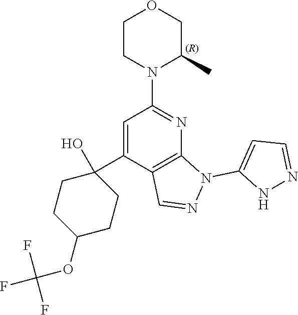 |
| 134 | 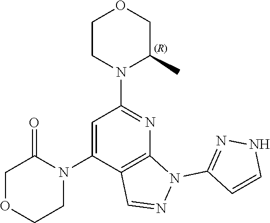 |
| 135 | 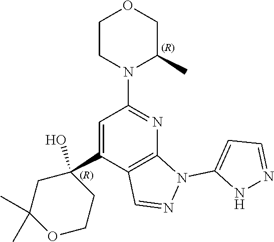 |
| 136 | 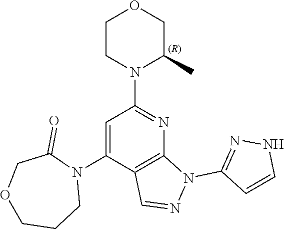 |
| 137 | 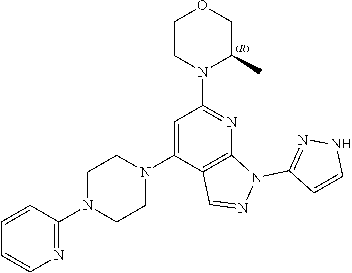 |
| 138 | 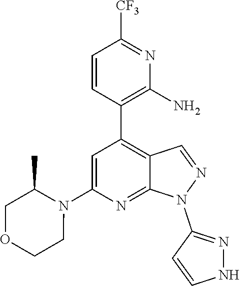 [SMILES: C[C@@H]1COCCN1c1cc(-c2ccc(C(F)(F)F)nc2N)c2cnn(-c3cc[nH]n3)c2n1] |
| 139 | 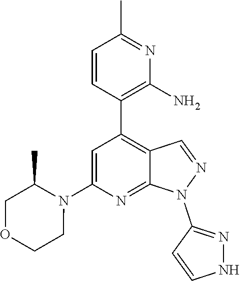 [SMILES: Cc1ccc(-c2cc(N3CCOC[C@H]3C)nc3c2cnn3-c2cc[nH]n2)c(N)n1] |
| 140 | 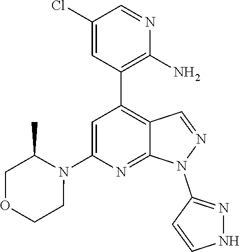 [SMILES: C[C@@H]1COCCN1c1cc(-c2cc(Cl)cnc2N)c2cnn(-c3cc[nH]n3)c2n1] |
| 141 | 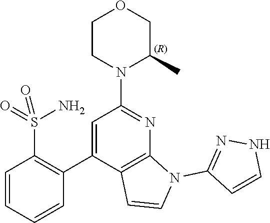 |
| 142 | 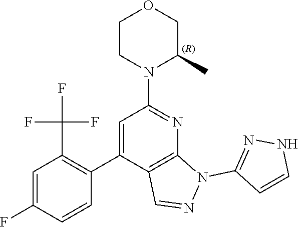 |
| 143 | 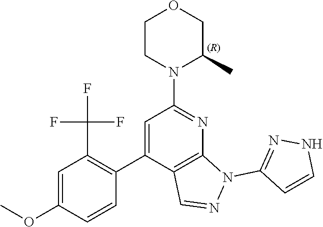 |
| 144 | 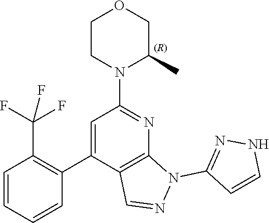 |
| 145 | 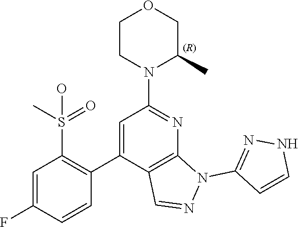 |
| 146 | 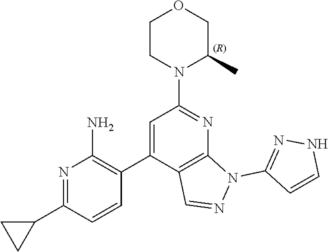 |
| 147 | 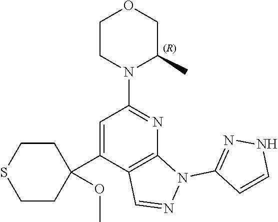 |
| 148 | 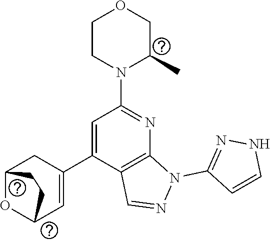 [SMILES: C[C@@H]1COCCN1c1cc(C2=C[C@H]3CC[C@@H](C2)O3)c2cnn(-c3cc[nH]n3)c2n1] |
| 149 | 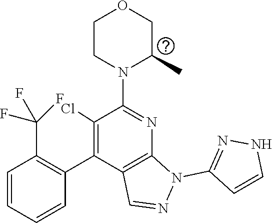 [SMILES: C[C@@H]1COCCN1c1nc2c(cnn2-c2cc[nH]n2)c(-c2ccccc2C(F)(F)F)c1Cl] |
| 150 | 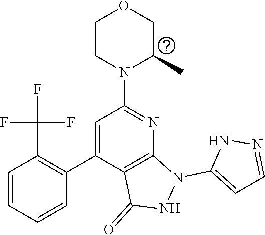 [SMILES: C[C@@H]1COCCN1c1cc(-c2ccccc2C(F)(F)F)c2c(=O)[nH]n(-c3ccn[nH]3)c2n1] |
| 151 | 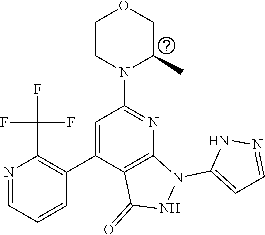 [SMILES: C[C@@H]1COCCN1c1cc(-c2cccnc2C(F)(F)F)c2c(=O)[nH]n(-c3ccn[nH]3)c2n1] |
| 152 | 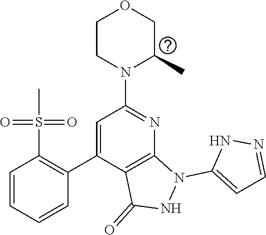 [SMILES: C[C@@H]1COCCN1c1cc(-c2ccccc2S(C)(=O)=O)c2c(=O)[nH]n(-c3ccn[nH]3)c2n1] |

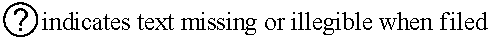

An ATR inhibitor may be isotopically enriched (e.g., enriched for deuterium).

PARP Inhibitors

A PARP inhibitor that may be used in the present invention may be a compound of formula (III):

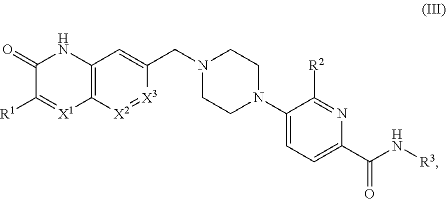

where
X¹ and X² are each independently selected from N and C(H),
X³ is independently selected from N and C(R⁴), where R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹ is C(H), and X³ is C(R⁴), and
when X³ is N, then X¹ and X² are both C(H).

R³ in formula (III) may be C₁₋₄ alkyl (e.g., methyl). R¹ in formula (III) may be ethyl.

The PARP inhibitor may be of formula (IIIa):

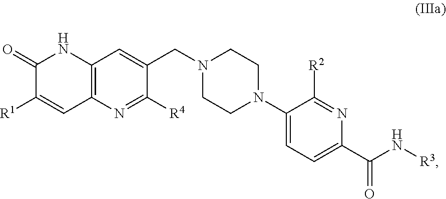

where
R¹ is C₁₋₄ alkyl,
R² is H, halo, C₁₋₄ alkyl, or C₁₋₄ fluoroalkyl,
R³ is H or C₁₋₄ alkyl, and
R⁴ is H.

R² may be difluoromethyl, trifluoromethyl, or methyl. R² may be H or halo. R¹ may be ethyl; R² may be H, chloro or fluoro; and R³ may be methyl.

The PARP inhibitor may be of formula (IIIb):

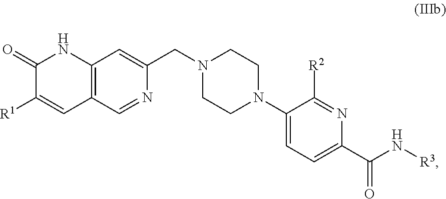

where
R¹ is C₁₋₄ alkyl,
R² is H or halo, and
R³ is H or C₁₋₄ alkyl.

R¹ may be ethyl; R² may be H, chloro, or fluoro; and R³ may be methyl.

The PARP inhibitor may be of formula (IIIc):

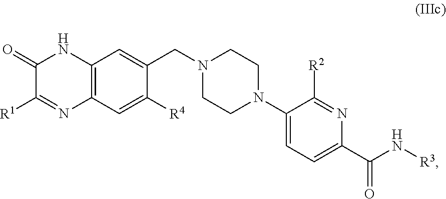

where
R¹ is C₁₋₄ alkyl or C₁₋₄ fluoroalkyl,
R² is H, halo, C₁₋₄ alkyl, or C₁₋₄ fluoroalkyl,
R³ is H or C₁₋₄ alkyl, and
R⁴ is H or fluoro.

R¹ may be ethyl, n-propyl, trifluoromethyl, 1,1-difluoroethyl, 2,2-difluroethyl, 2-fluoroethyl, or 2,2,2-trifluoroethyl. R² may be H, methyl, ethyl, trifluoromethyl, difluoromethyl, fluoromethyl, fluoro, or chloro. R³ may be H or methyl. In some embodiments, R⁴ is H.

The PARP inhibitor may be:
5-[4-[(3-ethyl-2-oxo-1H-1,6-naphthyridin-7-yl)methyl]piperazin-1-yl]-N-methyl-pyridine-2-carboxamide,
5-[4-[(3-ethyl-2-oxo-1H-1,6-naphthyridin-7-yl)methyl]piperazin-1-yl]-6-fluoro-N-methyl-pyridine-2-carboxamide,
6-chloro-5-[4-[(3-ethyl-2-oxo-1H-1,6-naphthyridin-7-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-6-fluoro-N-methyl-pyridine-2-carboxamide,
6-chloro-5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]pyridine-2-carboxamide,
6-ethyl-5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-N-methyl-6-(trifluoromethyl)pyridine-2-carboxamide,
6-(difluoromethyl)-5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-6-fluoro-N-methyl-pyridine-2-carboxamide,
5-[4-[(2-ethyl-3-oxo-4H-quinoxalin-6-yl)methyl]piperazin-1-yl]-N,6-dimethyl-pyridine-2-carboxamide,
6-chloro-5-[4-[(2-ethyl-3-oxo-4H-quinoxalin-6-yl)methyl]piperazin-1-yl]-N-methyl-pyridine-2-carboxamide,
N-methyl-5-[4-[[3-oxo-2-(trifluoromethyl)-4H-quinoxalin-6-yl]methyl]piperazin-1-yl]pyridine-2-carboxamide,
6-chloro-N-methyl-5-[4-[[3-oxo-2-(trifluoromethyl)-4H-quinoxalin-6-yl]methyl]piperazin-1-yl]pyridine-2-carboxamide,
6-fluoro-N-methyl-5-[4-[[3-oxo-2-(trifluoromethyl)-4H-quinoxalin-6-yl]methyl]piperazin-1-yl]pyridine-2-carboxamide,
N-methyl-5-[4-[(3-oxo-2-propyl-4H-quinoxalin-6-yl)methyl]piperazin-1-yl]pyridine-2-carboxamide,
6-chloro-N-methyl-5-[4-[(3-oxo-2-propyl-4H-quinoxalin-6-yl)methyl]piperazin-1-yl]pyridine-2-carboxamide,
6-fluoro-N-methyl-5-[4-[(3-oxo-2-propyl-4H-quinoxalin-6-yl)methyl]piperazin-1-yl]pyridine-2-carboxamide,
5-[4-[(2-ethyl-7-fluoro-3-oxo-4H-quinoxalin-6-yl)methyl]piperazin-1-yl]-6-fluoro-N-methyl-pyridine-2-carboxamide,
5-[4-[[2-(1,1-difluoroethyl)-3-oxo-4H-quinoxalin-6-yl]methyl]piperazin-1-yl]-N-methyl-pyridine-2-carboxamide,
5-[4-[[2-(2,2-difluoroethyl)-3-oxo-4H-quinoxalin-6-yl]methyl]piperazin-1-yl]-N-methyl-pyridine-2-carboxamide,
5-[4-[[2-(2,2-difluoroethyl)-3-oxo-4H-quinoxalin-6-yl]methyl]piperazin-1-yl]-6-fluoro-N-methyl-pyridine-2-carboxamide,
5-[4-[[2-(2-fluoroethyl)-3-oxo-4H-quinoxalin-6-yl]methyl]piperazin-1-yl]-N-methyl-pyridine-2-carboxamide,
6-fluoro-5-[4-[[2-(2-fluoroethyl)-3-oxo-4H-quinoxalin-6-yl]methyl]piperazin-1-yl]-N-methyl-pyridine-2-carboxamide,
N-methyl-5-[4-[[3-oxo-2-(2,2,2-trifluoroethyl)-4H-quinoxalin-6-yl]methyl]piperazin-1-yl]pyridine-2-carboxamide,
6-fluoro-N-methyl-5-(4-((3-oxo-2-(2,2,2-trifluoroethyl)-3,4-dihydroquinoxalin-6-yl)methyl) piperazin-1-yl)picolinamide,
   or a pharmaceutically acceptable salt thereof.

The PARP inhibitor may be 5-[4-[(7-ethyl-6-oxo-5H-1,5-naphthyridin-3-yl)methyl]piperazin-1-yl]-N-methyl-pyridine-2-carboxamide, or a pharmaceutically acceptable salt thereof.

The PARP inhibitor may be:
6-(difluoromethyl)-5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-N-methyl-6 (trifluoromethyl)pyridine-2-carboxamide,
5-[4-[(7-ethyl-6-oxo-5H-1,5-naphthyridin-3-yl)methyl]piperazin-1-yl]-N,6-dimethyl-pyridine-2-carboxamide,
N-ethyl-5-[4-[(7-ethyl-6-oxo-5H-1,5-naphthyridin-3-yl)methyl]piperazin-1-yl]pyridine-2-carboxamide,
   or a pharmaceutically acceptable salt thereof.

A PARP inhibitor that may be used in the present invention may be a compound of formula (IV):

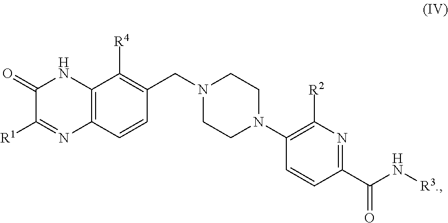

where
R¹ is independently selected from H, C₁₋₄ alkyl, C₃₋₆ cycloalkyl, C₁₋₄ fluoroalkyl, and C₁₋₄ alkyloxy;
R² is independently selected from H, halo, C₁₋₄ alkyl, and C₁₋₄ fluoroalkyl;
R³ is H or C₁₋₄ alkyl; and
R⁴ is halo or C₁₋₄ alkyl,
   or a pharmaceutically acceptable salt thereof.

The PARP inhibitor may be:
5-[4-[(2,5-dimethyl-3-oxo-4H-quinoxalin-6-yl)methyl]piperazin-1-yl]-6-fluoro-N-methyl-pyridine-2-carboxamide, 5-[4-[(2,5-dimethyl-3-oxo-4H-quinoxalin-6-yl)methyl]piperazin-1-yl]-N-methyl-pyridine-2-carboxamide,
6-chloro-5-[4-[(2,5-dimethyl-3-oxo-4H-quinoxalin-6-yl)methyl]piperazin-1-yl]-N-methyl-pyridine-2-carboxamide,
5-[4-[(2,5-dimethyl-3-oxo-4H-quinoxalin-6-yl)methyl]piperazin-1-yl]-N,6-dimethyl-pyridine-2-carboxamide,
5-[4-[(2,5-dimethyl-3-oxo-4H-quinoxalin-6-yl)methyl]piperazin-1-yl]-6-fluoro-pyridine-2-carboxamide,
5-[4-[(5-fluoro-2-methyl-3-oxo-4H-quinoxalin-6-yl)methyl]piperazin-1-yl]-6-methyl-pyridine-2-carboxamide,
5-[4-[(2,5-dimethyl-3-oxo-4H-quinoxalin-6-yl)methyl]piperazin-1-yl]-6-methyl-pyridine-2-carboxamide,
6-chloro-5-[4-[(5-chloro-2-ethyl-3-oxo-4H-quinoxalin-6-yl)methyl]piperazin-1-yl]-N-methyl-pyridine-2-carboxamide,
5-[4-[(5-chloro-2-ethyl-3-oxo-4H-quinoxalin-6-yl)methyl]piperazin-1-yl]-6-fluoro-N-methyl-pyridine-2-carboxamide,
5-[4-[(5-chloro-2-ethyl-3-oxo-4H-quinoxalin-6-yl)methyl]piperazin-1-yl]-N-methyl-pyridine-2-carboxamide,
5-[4-[(5-chloro-2-ethyl-3-oxo-4H-quinoxalin-6-yl)methyl]piperazin-1-yl]-N,6-dimethyl-pyridine-2-carboxamide,
6-fluoro-5-[4-[[5-fluoro-2-[(1S and 1R)-1-fluoroethyl]-3-oxo-4H-quinoxalin-6-yl]methyl]piperazin-1-yl]-N-methyl-pyridine-2-carboxamide,
5-[4-[[5-fluoro-2-[(1S and 1R)-1-fluoroethyl]-3-oxo-4H-quinoxalin-6-yl]methyl]piperazin-1-yl]-N,6-dimethyl-pyridine-2-carboxamide,
5-[4-[(5-chloro-2-methyl-3-oxo-4H-quinoxalin-6-yl)methyl]piperazin-1-yl]-N-methyl-pyridine-2-carboxamide,
5-[4-[(5-chloro-2-methyl-3-oxo-4H-quinoxalin-6-yl)methyl]piperazin-1-yl]-6-fluoro-N-methyl-pyridine-2-carboxamide,
5-[4-[(5-chloro-2-methyl-3-oxo-4H-quinoxalin-6-yl)methyl]piperazin-1-yl]-N,6-dimethyl-pyridine-2-carboxamide,
5-[4-[[2-(1,1-difluoroethyl)-5-fluoro-3-oxo-4H-quinoxalin-6-yl]methyl]piperazin-1-yl]-N, 6-dimethyl-pyridine-2-carboxamide,
6-fluoro-5-[4-[(5-fluoro-2-methyl-3-oxo-4H-quinoxalin-6-yl)methyl]piperazin-1-yl]-N-methyl-pyridine-2-carboxamide, 6-(difluoromethyl)-5-[4-[(5-fluoro-2-methyl-3-oxo-4H-quinoxalin-6-yl)methyl]piperazin-1-yl]-N-methyl-pyridine-2-carboxamide,
6-fluoro-5-[4-[(5-fluoro-2-methyl-3-oxo-4H-quinoxalin-6-yl)methyl]piperazin-1-yl]pyridine-2-carboxamide,
5-[4-[(2-ethyl-5-fluoro-3-oxo-4H-quinoxalin-6-yl)methyl]piperazin-1-yl]-N,6-dimethyl-pyridine-2-carboxamide,
6-(difluoromethyl)-5-[4-[(2-ethyl-5-fluoro-3-oxo-4H-quinoxalin-6-yl)methyl]piperazin-1-yl]-N-methyl-pyridine-2-carboxamide,
5-[4-[(2-ethyl-5-fluoro-3-oxo-4H-quinoxalin-6-yl)methyl]piperazin-1-yl]pyridine-2-carboxamide,
5-[4-[(2-ethyl-5-fluoro-3-oxo-4H-quinoxalin-6-yl)methyl]piperazin-1-yl]-6-methyl-pyridine-2-carboxamide,
5-[4-[(2-ethyl-5-fluoro-3-oxo-4H-quinoxalin-6-yl)methyl]piperazin-1-yl]-6-fluoro-N-methyl-pyridine-2-carboxamide,
6-chloro-5-[4-[(2-ethyl-5-fluoro-3-oxo-4H-quinoxalin-6-yl)methyl]piperazin-1-yl]-N-methyl-pyridine-2-carboxamide,
5-[4-[(2-ethyl-5-fluoro-3-oxo-4H-quinoxalin-6-yl)methyl]piperazin-1-yl]-N-methyl-pyridine-2-carboxamide,
6-chloro-5-[4-[(5-fluoro-2-methyl-3-oxo-4H-quinoxalin-6-yl)methyl]piperazin-1-yl]-N-methyl-pyridine-2-carboxamide,
5-[4-[(5-fluoro-2-methyl-3-oxo-4H-quinoxalin-6-yl)methyl]piperazin-1-yl]-N,6-dimethyl-pyridine-2-carboxamide,
5-[4-[(5-fluoro-2-methyl-3-oxo-4H-quinoxalin-6-yl)methyl]piperazin-1-yl]-N-methyl-pyridine-2-carboxamide,
5-[4-[(5-fluoro-3-oxo-4H-quinoxalin-6-yl)methyl]piperazin-1-yl]-N-methyl-pyridine-2-carboxamide,
6-chloro-5-[4-[(5-fluoro-3-oxo-4H-quinoxalin-6-yl)methyl]piperazin-1-yl]-N-methyl-pyridine-2-carboxamide,
5-[4-[(5-fluoro-3-oxo-4H-quinoxalin-6-yl)methyl]piperazin-1-yl]-N,6-dimethyl-pyridine-2-carboxamide,
6-fluoro-5-[4-[(5-fluoro-3-oxo-4H-quinoxalin-6-yl)methyl]piperazin-1-yl]-N-methyl-pyridine-2-carboxamide,
5-[4-[[2-(difluoromethyl)-5-fluoro-3-oxo-4H-quinoxalin-6-yl]methyl]piperazin-1-yl]-N, 6-dimethyl-pyridine-2-carboxamide, 5-[4-[(5-fluoro-2-methoxy-3-oxo-4H-quinoxalin-6-yl)methyl]piperazin-1-yl]-N-methyl-pyridine-2-carboxamide,
6-fluoro-5-[4-[(5-fluoro-2-methoxy-3-oxo-4H-quinoxalin-6-yl)methyl]piperazin-1-yl]-N-methyl-pyridine-2-carboxamide,
5-[4-[(5-fluoro-2-methoxy-3-oxo-4H-quinoxalin-6-yl)methyl]piperazin-1-yl]-N,6-dimethyl-pyridine-2-carboxamide,
6-chloro-5-[4-[(5-fluoro-2-methoxy-3-oxo-4H-quinoxalin-6-yl)methyl]piperazin-1-yl]-N-methyl-pyridine-2-carboxamide,
5-[4-[(2-ethyl-5-methyl-3-oxo-4H-quinoxalin-6-yl)methyl]piperazin-1-yl]-N,6-dimethyl-pyridine-2-carboxamide,
5-[4-[(2-ethyl-5-methyl-3-oxo-4H-quinoxalin-6-yl)methyl]piperazin-1-yl]-6-fluoro-N-methyl-pyridine-2-carboxamide,
5-[4-[(2-ethyl-5-methyl-3-oxo-4H-quinoxalin-6-yl)methyl]piperazin-1-yl]-N-methyl-pyridine-2-carboxamide,
N-ethyl-6-fluoro-5-[4-[(5-fluoro-2-methyl-3-oxo-4H-quinoxalin-6-yl)methyl]piperazin-1-yl]pyridine-2-carboxamide,
N-ethyl-5-[4-[(5-fluoro-2-methyl-3-oxo-4H-quinoxalin-6-yl)methyl]piperazin-1-yl]-6-methyl-pyridine-2-carboxamide,
5-[4-[[5-fluoro-3-oxo-2-(trifluoromethyl)-4H-quinoxalin-6-yl]methyl]piperazin-1-yl]-N, 6-dimethyl-pyridine-2-carboxamide,
6-fluoro-5-[4-[[5-fluoro-3-oxo-2-(trifluoromethyl)-4H-quinoxalin-6-yl]methyl]piperazin-1-yl]-N-methyl-pyridine-2-carboxamide,
6-chloro-5-[4-[[5-fluoro-3-oxo-2-(trifluoromethyl)-4H-quinoxalin-6-yl]methyl]piperazin-1-yl]-N-methyl-pyridine-2-carboxamide,
5-[4-[[5-fluoro-3-oxo-2-(trifluoromethyl)-4H-quinoxalin-6-yl]methyl]piperazin-1-yl]-N-methyl-pyridine-2-carboxamide,
6-fluoro-5-[4-[(5-fluoro-2-isopropyl-3-oxo-4H-quinoxalin-6-yl)methyl]piperazin-1-yl]-N-methyl-pyridine-2-carboxamide,
5-[4-[(5-fluoro-2-isopropyl-3-oxo-4H-quinoxalin-6-yl)methyl]piperazin-1-yl]-N,6-dimethyl-pyridine-2-carboxamide,
5-[4-[(5-fluoro-2-isopropyl-3-oxo-4H-quinoxalin-6-yl)methyl]piperazin-1-yl]-N-methyl-pyridine-2-carboxamide, 5-[4-[(2-cyclopropyl-5-fluoro-3-oxo-4H-quinoxalin-6-yl)methyl]piperazin-1-yl]-6-fluoro-N-methyl-pyridine-2-carboxamide,
5-[4-[(2-cyclopropyl-5-fluoro-3-oxo-4H-quinoxalin-6-yl)methyl]piperazin-1-yl]-N,6-dimethyl-pyridine-2-carboxamide,
5-[4-[(2-cyclopropyl-5-fluoro-3-oxo-4H-quinoxalin-6-yl)methyl]piperazin-1-yl]-N-methyl-pyridine-2-carboxamide,
5-[4-[(2-methoxy-5-methyl-3-oxo-4H-quinoxalin-6-yl)methyl]piperazin-1-yl]-N,6-dimethyl-pyridine-2-carboxamide,
6-fluoro-5-[4-[(2-methoxy-5-methyl-3-oxo-4H-quinoxalin-6-yl)methyl]piperazin-1-yl]-N-methyl-pyridine-2-carboxamide,
6-(difluoromethyl)-5-[4-[(2-methoxy-5-methyl-3-oxo-4H-quinoxalin-6-yl)methyl]piperazin-1-yl]-N-methyl-pyridine-2-carboxamide,
6-(difluoromethyl)-5-[4-[(2,5-dimethyl-3-oxo-4H-quinoxalin-6-yl)methyl]piperazin-1-yl]-N-methyl-pyridine-2-carboxamide, or
   a pharmaceutically acceptable salts thereof.

The PARP inhibitor may be PARP inhibitor is 6-fluoro-5-[4-[(5-fluoro-2-methyl-3-oxo-4H-quinoxalin-6-yl)methyl]piperazin-1-yl]-N-methyl-pyridine-2-carboxamide, or a pharmaceutically acceptable salt thereof.

The PARP inhibitor may be 6-fluoro-5-[4-[(5-fluoro-2-methyl-3-oxo-4H-quinoxalin-6-yl)methyl]piperazin-1-yl]-N-methyl-pyridine-2-carboxamide.

The PARP inhibitor may be isotopically enriched (e.g., enriched for deuterium).

The PARP inhibitors may be prepared as described in US 2021/0040084 and US 2022/0009901, the disclosures of which are incorporated herein in their entirety.

Alternatively, the PARP inhibitor used in the methods of the invention may be veliparib (ABT-888), iniparib (BSI-201), 2X-121, CEP-9722, KU-0059436 (AZD2281), PF-01367338, a pharmaceutically acceptable salt thereof, or a combination thereof.

Isomers and Compositions Thereof

The invention includes (where possible) individual diastereomers, enantiomers, epimers, and atropisomers of the compounds disclosed herein, and mixtures of diastereomers and/or enantiomers thereof including racemic mixtures. Although the specific stereochemistries disclosed herein are preferred, other stereoisomers, including diastereomers, enantiomers, epimers, atropisomers, and mixtures of these may also have utility in treating diseases. Inactive or less active diastereoisomers and enantiomers may be useful, e.g., for scientific studies relating to the receptor and the mechanism of activation.

It is understood that certain molecules can exist in multiple tautomeric forms. This invention includes all tautomers even though only one tautomer may be indicated in the examples.

The invention also includes pharmaceutically acceptable salts of the compounds, and pharmaceutical compositions including the compounds and a pharmaceutically acceptable carrier. The compounds are especially useful, e.g., in certain kinds of cancer and for slowing the progression of cancer once it has developed in a patient.

The compounds disclosed herein may be used in pharmaceutical compositions including (a) the compound(s) or pharmaceutically acceptable salts thereof, and (b) a pharmaceutically acceptable carrier. The compounds may be used in pharmaceutical compositions that include one or more other active pharmaceutical ingredients. The compounds may also be used in pharmaceutical compositions in which the compound disclosed herein or a pharmaceutically acceptable salt thereof is the only active ingredient.

Optical Isomers—Diastereomers—Geometric Isomers—Tautomers

Compounds disclosed herein may contain, e.g., one or more stereogenic centers and can occur as racemates, racemic mixtures, single enantiomers, individual diastereomers, and mixtures of diastereomers and/or enantiomers. The invention includes all such isomeric forms of the compounds disclosed herein. It is intended that all possible stereoisomers (e.g., enantiomers and/or diastereomers) in mixtures and as pure or partially purified compounds are included within the scope of this invention (i.e., all possible combinations of the stereogenic centers as pure compounds or in mixtures).

Some of the compounds described herein may contain bonds with hindered rotation such that two separate rotomers, or atropisomers, may be separated and found to have different biological activity which may be advantageous. It is intended that all of the possible atropisomers are included within the scope of this invention.

Some of the compounds described herein may contain olefinic double bonds, and unless specified otherwise, are meant to include both E and Z geometric isomers.

Some of the compounds described herein may exist with different points of attachment of hydrogen, referred to as tautomers. An example is a ketone and its enol form, known as keto-enol tautomers. The individual tautomers as well as mixtures thereof are encompassed by the invention.

Compounds disclosed herein having one or more asymmetric centers may be separated into diastereoisomers, enantiomers, and the like by methods well known in the art.

Alternatively, enantiomers and other compounds with chiral centers may be synthesized by stereospecific synthesis using optically pure starting materials and/or reagents of known configuration.

Metabolites—Prodrugs

The invention includes therapeutically active metabolites, where the metabolites themselves fall within the scope of the claims. The invention also includes prodrugs, which are compounds that are converted to the claimed compounds as they are being administered to a patient or after they have been administered to a patient. The claimed chemical structures of this application in some cases may themselves be prodrugs.

Isotopically Enriched Derivatives

The invention includes molecules which have been isotopically enriched at one or more position within the molecule. Thus, compounds enriched for deuterium fall within the scope of the claims.

Methods of Preparing ATR Inhibitors and PARP inhibitors

ATR inhibitors may be prepared using reactions and techniques known in the art. For example, certain ATR inhibitors may be prepared using techniques and methods disclosed in, e.g., International Application Nos. PCT/US2019/051539 and PCT/US2018/034729, each of which is incorporated by reference herein; U.S. Pat. Nos. 9,663,535, 9,549,932, 8,552,004, and 8,841,308, each of which is incorporated by reference herein; and U.S. Patent Application Publication No. 2019/0055240, which is incorporated by reference herein.

PARP inhibitors may be prepared using reactions and techniques known in the art. For example, certain PARP inhibitors may be prepared using techniques and methods disclosed in, e.g., U.S. Pat. Nos. 8,716,493, 8,236,802, 8,071,623, 8,012,976, 7,732,491, 7,550,603, 7,531,530, 7,151,102, and 6,495,541, each of which is incorporated herein by reference herein.

Methods of Use

ATR inhibitors and PARP inhibitors may be used together for the treatment of a disease or condition having the symptom of cell hyperproliferation. For example, the invention described herein may be applicable for treatment of various oncological conditions harboring sensitizing gene mutations, such as tumors with any deleterious (loss-of-function) alterations in ATM, BRCA2, RNASEH2A, RNASEH2B, and CDK12. In particular, mutations in one or more of these genes may be frequently found in the following tumor types: renal cell carcinoma, mature B-cell neoplasms, endometrial cancer, ovarian cancer, colorectal cancer, skin cancer (non-melanoma), small bowel cancer, non-small cell lung cancer, melanoma, bladder cancer, pancreatic cancer, head and neck cancer, mesothelioma, glioma, prostate cancer, breast cancer, and esophagogastric cancer. Accordingly, methods of the invention are preferably used in the treatment of these cancers.

Therapeutic methods of the invention include the step of administering a therapeutically effective amount of an ATR inhibitor and a therapeutically effective amount of a PARP inhibitor to a subject in need thereof. The therapeutically effective amount of a PARP inhibitor may be, e.g., a subtherapeutic regimen of a PARP inhibitor. The therapeutically effective amount of an ATR inhibitor may be, e.g., a subtherapeutic regimen of an ATR inhibitor.

The disease or condition treated using methods of the invention may have the symptom of cell hyperproliferation. For example, the disease or condition may be a cancer. The cancer may be, e.g., carcinoma, sarcoma, adenocarcinoma, lymphoma, leukemia, or melanoma. The cancer may be, e.g., a solid tumor.

Non-limiting examples of cancers include prostate cancer, breast cancer, ovarian cancer, multiple myeloma, brain cancer, glioma, lung cancer, salivary cancer, stomach cancer, thymic epithelial cancer, thyroid cancer, leukemia, melanoma, lymphoma, gastric cancer, pancreatic cancer, kidney cancer, bladder cancer, colon cancer, and liver cancer.

Preferably, methods of the invention are used in the treatment of renal cell carcinoma, mature B-cell neoplasms, endometrial cancer, ovarian cancer, fallopian tube cancer, primary peritoneal cancer, colorectal cancer, skin cancer (non-melanoma), small bowel cancer, non-small cell lung cancer, melanoma, bladder cancer, pancreatic cancer, head and neck cancer, mesothelioma, glioma, prostate cancer, breast cancer, or esophagogastric cancer.

Non-limiting examples of carcinomas include medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifomi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Non-limiting examples of sarcomas include chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Non-limiting examples of leukemias include acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

Non-limiting examples of melanomas include acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

Pharmaceutical Compositions

The compounds used in the methods described herein are preferably formulated into pharmaceutical compositions for administration to human subjects in a biologically compatible form suitable for administration in vivo. Pharmaceutical compositions typically include a compound as described herein and a pharmaceutically acceptable excipient. Certain pharmaceutical compositions may include one or more additional pharmaceutically active agents described herein.

The compounds described herein can also be used in the form of the free base, in the form of salts, zwitterions, solvates, or as prodrugs, or pharmaceutical compositions thereof. All forms are within the scope of the invention. The compounds, salts, zwitterions, solvates, prodrugs, or pharmaceutical compositions thereof, may be administered to a patient in a variety of forms depending on the selected route of administration, as will be understood by those skilled in the art. The compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, a compound of the invention can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of a compound of the invention into preparations which can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., 40 mesh.

Dosages

The dosage of the compound used in the methods described herein, or pharmaceutically acceptable salts or prodrugs thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

A compound of the invention may be administered to the patient in a single dose or in multiple doses. When multiple doses are administered, the doses may be separated from one another by, for example, 1-24 hours, 1-7 days, 1-4 weeks, or 1-12 months. The compound may be administered according to a schedule or the compound may be administered without a predetermined schedule. An active compound may be administered, for example, 1, 2, 3, 4, 5, 6, 7, 8, 9, 10, 11, or 12 times per day, every 2nd, 3rd, 4th, 5th, or 6th day, 1, 2, 3, 4, 5, 6, or 7 times per week, 1, 2, 3, 4, 5, or 6 times per month, or 1, 2, 3, 4, 5, 6, 7, 8, 9, 10, 11, or 12 times per year. It is to be understood that, for any particular subject, specific dosage regimes should be adjusted over time according to the individual need and the professional judgment of the person administering or supervising the administration of the compositions.

While the attending physician ultimately will decide the appropriate amount and dosage regimen, an effective amount of a compound of the invention may be, for example, a total daily dosage of, e.g., between 0.05 mg and 3000 mg of any of the compounds described herein. Alternatively, the dosage amount can be calculated using the body weight of the patient. Such dose ranges may include, for example, between 0.05-1000 mg (e.g., 0.25-800 mg). In some embodiments, 0.05, 0.1, 0.25, 0.5, 1, 2, 3, 4, 5, 10, 15, 20, 25, 30, 35, 40, 45, 50, 100, 150, 200, 250, 300, 350, 400, 450, 500, 550, 600, 650, 700, 750, 800, 850, 900, 950, or 1000 mg of the compound is administered.

Preferably, the subtherapeutic regimen of an ATR inhibitor is a low dosage (e.g., at least 10%, 20%, 50%, 80%, 90%, or 95% less than the lowest standard recommended dosage of the ATR inhibitor for a given route of administration). Preferably, the dosage of a PARP inhibitor is a low dosage (e.g., at least 10%, 20%, 50%, 80%, 90%, or 95% less than the lowest standard recommended dosage of the PARP inhibitor for a given route of administration). Preferably, the ATR inhibitor is administered once daily or twice daily. The PARP inhibitor may be administered once daily, twice daily, 1 day/week, 2 days/week, 3 days/week, or 4 days/week. Preferably, the PARP inhibitor may be administered 1 day/week, 2 days/week, 3 days/week, or 4 days/week.

In the methods of the invention, the time period during which multiple doses of a compound of the invention are administered to a patient can vary. For example, in some embodiments, doses of the compounds of the invention are administered to a patient over a time period that is 1-7 days; 1-12 weeks; or 1-3 months. In other embodiments, the compounds are administered to the patient over a time period that is, for example, 4-11 months or 1-30 years. In other embodiments, the compounds are administered to a patient at the onset of symptoms. In any of these embodiments, the amount of compound that is administered may vary during the time period of administration. When a compound is administered daily, administration may occur, for example, 1, 2, or 3 times per day.

Formulations

A compound identified as capable of treating any of the conditions described herein, using any of the methods described herein, may be administered to patients or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to subjects in need thereof. Administration may begin before the patient is symptomatic.

Exemplary routes of administration of the compounds (e.g., a compound of the invention), or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The compounds desirably are administered with a pharmaceutically acceptable carrier. Pharmaceutical formulations of the compounds described herein formulated for treatment of the disorders described herein are also part of the present invention. Oral administration is a preferred route of administration in the methods of the invention.

Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution- or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, camauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

Formulations for Parenteral Administration

The compounds described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) “Drug Injection:” a liquid preparation that is a drug substance (e.g., a compound of the invention), or a solution thereof;
(2) “Drug for Injection:” the drug substance (e.g., a compound of the invention) as a dry solid that will be combined with the appropriate sterile vehicle for parenteral administration as a drug injection;
(3) “Drug Injectable Emulsion:” a liquid preparation of the drug substance (e.g., a compound of the invention) that is dissolved or dispersed in a suitable emulsion medium;
(4) “Drug Injectable Suspension:” a liquid preparation of the drug substance (e.g., a compound of the invention) suspended in a suitable liquid medium; and
(5) “Drug for Injectable Suspension:” the drug substance (e.g., a compound of the invention) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21st Ed., Gennaro, Ed., Lippincott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

The following examples are meant to illustrate the invention. They are not meant to limit the invention in any way.

### EXAMPLES

Synergy between ATR inhibitors and PARP inhibitors in various cancer cell line backgrounds

An ATR inhibitor (e.g., compound 121) may synergize with a PARP inhibitor (e.g., 5-[4-[(7-ethyl-6-oxo-5H-1,5-naphthyridin-3-yl)methyl]piperazin-1-yl]-N-methyl-pyridine-2-carboxamide) in RNASEH2B+/+ and RNASEH2B−/− cells. Importantly, in RNASEH2B−/− cells, synergy (e.g., maximum synergy) may be achieved at lower concentrations of an ATR inhibitor (e.g., compound 121) and lower concentrations of the PARP inhibitor (e.g., 5-[4-[(7-ethyl-6-oxo-5H-1,5-naphthyridin-3-yl)methyl]piperazin-1-yl]-N-methyl-pyridine-2-carboxamide) than in RNASEH2B+/+ cells. As a result, the apparent IC₅₀ of the PARP inhibitor may shift in the presence of the ATR inhibitor (e.g., compound 121).

In addition to selecting a sensitized genetic background, tolerability of the combination treatment with an ATR inhibitor and PARP inhibitor can be improved by optimizing dosing schedules (Fang et al., Cancer Cell, 35:851-867, 2019). To guide the combined dosing of the ATR inhibitor (e.g., compound 121) and the PARP inhibitor (e.g., 5-[4-[(7-ethyl-6-oxo-5H-1,5-naphthyridin-3-yl)methyl]piperazin-1-yl]-N-methyl-pyridine-2-carboxamide) in RNASEH2B-deficient tumor cells, a continuous longer (e.g., 168 h) concomitant treatment of RNASEH2B+/+ and −/− cells with both compounds may be compared to a shorter (e.g., either a 48 or 72 h) treatment followed by removal of compounds and growth in drug-free media. The presence of the ATR inhibitor (e.g., compound 121) may decrease the apparent IC₅₀ value of the PARP inhibitor (e.g., 5-[4-[(7-ethyl-6-oxo-5H-1,5-naphthyridin-3-yl)methyl]piperazin-1-yl]-N-methyl-pyridine-2-carboxamide) when dosed continuously for a longer period of time (e.g., 168 h). No significant difference in the apparent IC₅₀ values might be observed when treatment is shortened (e.g., to 72 or 48 h), suggesting that an intermittent dosing schedule of the ATR inhibitor (e.g., compound 121) in combination with the PARP inhibitor (e.g., 5-[4-[(7-ethyl-6-oxo-5H-1,5-naphthyridin-3-yl)methyl]piperazin-1-yl]-N-methyl-pyridine-2-carboxamide) followed by a recovery period may be efficacious in RNASEH2B-deficient cells. If the above results are observed, they would suggest that tumor cells of a specific genetic makeup, such as cells lacking RNASEH2B, can be treated with reduced doses of the PARP and ATR inhibitors for reduced amounts of time as compared to standard therapeutic regimens, while maintaining efficacy.

The optimized dosing schedule may be applicable to cells carrying mutations not only in RNASEH2B, but additional genes. For example, a 48 h treatment with a combination of the PARP inhibitor (e.g., 5-[4-[(7-ethyl-6-oxo-5H-1,5-naphthyridin-3-yl)methyl]piperazin-1-yl]-N-methyl-pyridine-2-carboxamide) and the ATR inhibitor (e.g., compound 121) may synergistically sensitize (>300×) an ATM CRISPR knockout clone (ATM−/−) of a cancer cell line (e.g., the MIAPACA2 pancreatic carcinoma cell line). Furthermore, a 48-hr treatment with a combination of the ATR inhibitor (e.g., compound 121) and the PARP inhibitor (e.g., 5-[4-[(7-ethyl-6-oxo-5H-1,5-naphthyridin-3-yl)methyl]piperazin-1-yl]-N-methyl-pyridine-2-carboxamide) may synergistically sensitize isogenic cell lines lacking CDK12 or BRCA2.

In addition to tumors carrying the above-mentioned genetic alterations, the combination of ATR inhibitors (e.g., Compound 121) with PARP inhibitors (e.g., 5-[4-[(7-ethyl-6-oxo-5H-1,5-naphthyridin-3-yl)methyl]piperazin-1-yl]-N-methyl-pyridine-2-carboxamide) may be effective against tumor cells that employ the Alternative Lengthening of Telomeres (ALT) mechanism.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of treating a cancer in a subject, the method comprising administering to the subject in need thereof a therapeutically effective amount of an ATR inhibitor and a therapeutically effective amount of a PARP inhibitor, wherein the cancer has been previously identified as a cancer having a loss of function of ATM, BRCA2, RNAse H2A, RNAse H2B, CDK12, or a combination thereof, or wherein the cancer has been previously identified as an ALT+ cancer,
and wherein the PARP inhibitor is a compound of formula (III) or (IV): 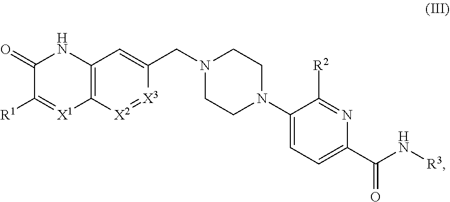
wherein
X¹ and X² are each independently selected from N and C(H),
X³ is independently selected from N and C(R⁴), wherein R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹ is C(H), and X³ is C(R⁴), and
when X³ is N, then X¹ and X² are both C(H);
or 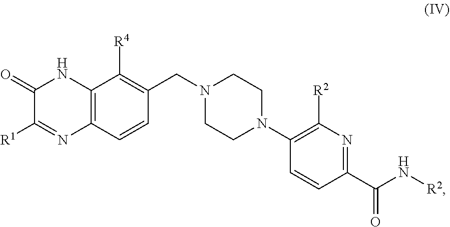
wherein
R¹ is independently selected from H, C₁₋₄ alkyl, C₃₋₆ cycloalkyl, C₁₋₄ fluoroalkyl, and C₁₋₄ alkyloxy;
R² is independently selected from H, halo, C₁₋₄ alkyl, and C₁₋₄ fluoroalkyl;
R³ is H or C₁₋₄ alkyl; and
R⁴ is halo or C₁₋₄ alkyl,
or a pharmaceutically acceptable salt thereof.

2. A method of treating a cancer in a subject, the method comprising administering to the subject in need thereof a therapeutically effective amount of an ATR inhibitor and a therapeutically effective amount of a PARP inhibitor, wherein the cancer has a loss of function of ATM, BRCA2, RNAse H2A, RNAse H2B, CDK12, or a combination thereof, or wherein the cancer is an ALT+ cancer;
and wherein the PARP inhibitor is a compound of formula (III) or (IV): 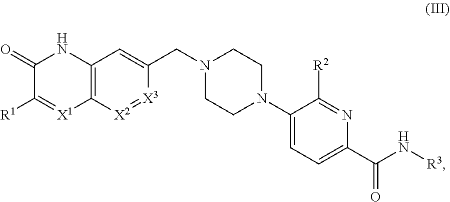
wherein
X¹ and X² are each independently selected from N and C(H),
X³ is independently selected from N and C(R⁴), wherein R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹ is C(H), and X³ is C(R⁴), and
when X³ is N, then X¹ and X² are both C(H);
or 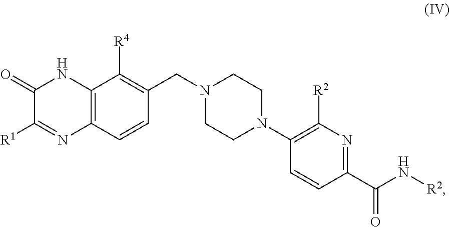
wherein
R¹ is independently selected from H, C₁₋₄ alkyl, C₃₋₆ cycloalkyl, C₁₋₄ fluoroalkyl, and C₁₋₄ alkyloxy;
R² is independently selected from H, halo, C₁₋₄ alkyl, and C₁₋₄ fluoroalkyl;
R³ is H or C₁₋₄ alkyl; and
R⁴ is halo or C₁₋₄ alkyl,
or a pharmaceutically acceptable salt thereof.

3. A method of treating a cancer in a subject, the method comprising:
(i) identifying the cancer as having a loss of function of ATM, BRCA2, RNAse H2A, RNAse H2B, CDK12, or a combination thereof, or as an ALT+ cancer; and
(ii) administering to the subject in need thereof a therapeutically effective amount of an ATR inhibitor and a therapeutically effective amount of a PARP inhibitor of formula (III) or (IV): 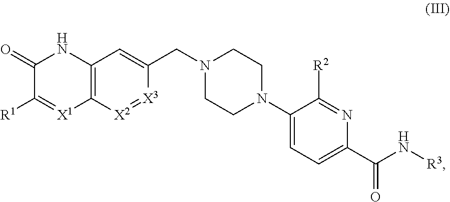
wherein
X¹ and X² are each independently selected from N and C(H),
X³ is independently selected from N and C(R⁴), wherein R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹ is C(H), and X³ is C(R⁴), and
when X³ is N, then X¹ and X² are both C(H);
or 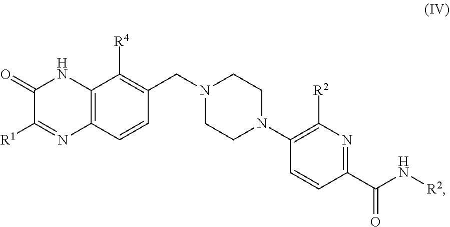
wherein
R¹ is independently selected from H, C₁₋₄ alkyl, C₃₋₆ cycloalkyl, C₁₋₄ fluoroalkyl, and C₁₋₄ alkyloxy;
R² is independently selected from H, halo, C₁₋₄ alkyl, and C₁₋₄ fluoroalkyl;
R³ is H or C₁₋₄ alkyl; and
R⁴ is halo or C₁₋₄ alkyl,
or a pharmaceutically acceptable salt thereof.

4. The method of any one of claims 1 to 3, wherein the ATR inhibitor is administered before the PARP inhibitor.

5. The method of any one of claims 1 to 3, wherein the ATR inhibitor is administered after the PARP inhibitor.

6. The method of any one of claims 1 to 3, wherein the ATR inhibitor is co-administered with the PARP inhibitor.

7. The method of any one of claims 1 to 6, wherein the therapeutically effective amount is a subtherapeutic regimen of the ATR inhibitor.

8. The method of any one of claims 1 to 7, wherein the therapeutically effective amount is a subtherapeutic regimen of the PARP inhibitor.

9. The method of claim 7 or 8, wherein the subtherapeutic regimen comprises a starting dosage that is at least 50% less than the lowest standard starting dosage that is used for a monotherapy.

10. The method of any one of claims 7 to 9, wherein the subtherapeutic regimen comprises a maintenance dosage that is at least 50% less than the lowest standard maintenance dosage that is used for a monotherapy.

11. The method of claim 10, wherein the maintenance dosage comprises a first reduced dosage.

12. The method of claim 10 or 11, wherein the maintenance dosage comprises a second reduced dosage.

13. The method of any one of claims 10 to 12, wherein the maintenance dosage comprises a third reduced dosage.

14. The method of any one of claims 1 to 13, wherein the route of administration is an oral administration.

15. The method of any one of claims 1 to 14, wherein the ATR inhibitor is administered 1 day/week, 2 days/week, or 3 days/week.

16. The method of any one of claims 1 to 15, wherein the PARP inhibitor is administered 1 day/week, 2 days/week, 3 days/week, or 4 days/week.

17. A method of inducing cell death in an aberrant cancer cell having a loss of function of ATM, BRCA2, RNAse H2A, RNAse H2B, CDK12, or a combination thereof, or in an ALT+ cancer cell, the method comprising contacting the cell with an effective amount of an ATR inhibitor and an effective amount of a PARP inhibitor, the effective amounts being sufficient to induce cell death in the aberrant cancer cell;
and wherein the PARP inhibitor is a compound of formula (III) or (IV): 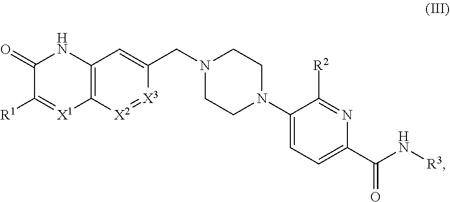
wherein
X¹ and X² are each independently selected from N and C(H),
X³ is independently selected from N and C(R⁴), wherein R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹ is C(H), and X³ is C(R⁴), and
when X³ is N, then X¹ and X² are both C(H);
or 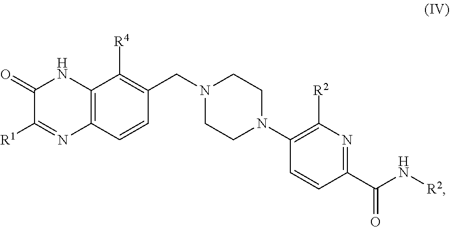
wherein
R¹ is independently selected from H, C₁₋₄ alkyl, C₃₋₆ cycloalkyl, C₁₋₄ fluoroalkyl, and C₁₋₄ alkyloxy;
R² is independently selected from H, halo, C₁₋₄ alkyl, and C₁₋₄ fluoroalkyl;
R³ is H or C₁₋₄ alkyl; and
R⁴ is halo or C₁₋₄ alkyl,
or a pharmaceutically acceptable salt thereof.

18. The method of any one of claims 1 to 17, wherein the loss of function is a loss of function of ATM.

19. The method of any one of claims 1 to 17, wherein the loss of function is a loss of function of RNAse H2A.

20. The method of any one of claims 1 to 17, wherein the loss of function is a loss of function of RNAse H2B.

21. The method of any one of claims 1 to 17, wherein the loss of function is a loss of function of CDK12.

22. The method of any one of claims 1 to 17, wherein the loss of function is a loss of function of BRCA2.

23. The method of any one of claims 1 to 17, wherein the cancer is an ALT+ cancer.

24. The method of any one of claims 1 to 23, wherein the ATR inhibitor is a compound of formula (I): 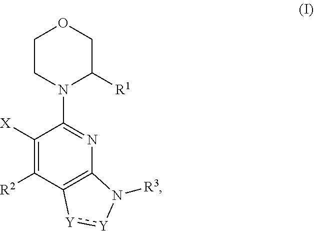
or a pharmaceutically acceptable salt thereof,
wherein [symbol]
is a double bond, and each Y is independently N or CR⁴; or [symbol] is a single bond, and each Y is independently NR^{Y}, carbonyl, or C(R^{Y})₂; wherein each R^{Y} is independently H or optionally substituted C₁₋₆ alkyl;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

25. The method of claim 24, wherein the ATR inhibitor is a compound of formula (II): 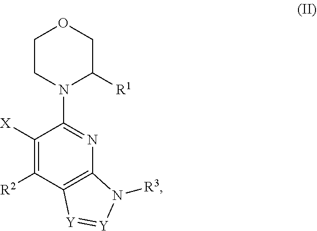
or a pharmaceutically acceptable salt thereof,
wherein
each Y is independently N or CR⁴;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

26. The method of claim 24, wherein the ATR inhibitor is selected from the group consisting of compounds 43, 57, 62, 87, 93, 94, 95, 99, 100, 106, 107, 108, 109, 111, 112, 113, 114, 115, 116, 118, 119, 120, 121, 122, 123, 135, 147, 148, and pharmaceutically acceptable salts thereof.

27. The method of claim 26, wherein the ATR inhibitor is compound 43 or a pharmaceutically acceptable salt thereof.

28. The method of claim 26, wherein the ATR inhibitor is compound 121 or a pharmaceutically acceptable salt thereof.

29. The method of claim 26, wherein the ATR inhibitor is compound 122 or a pharmaceutically acceptable salt thereof.

30. The method of any one of claims 1 to 29, wherein the cancer is renal cell carcinoma, mature B-cell neoplasms, endometrial cancer, ovarian cancer, fallopian tube cancer, primary peritoneal cancer, colorectal cancer, skin cancer, small bowel cancer, non-small cell lung cancer, melanoma, bladder cancer, pancreatic cancer, head and neck cancer, mesothelioma, glioma, prostate cancer, breast cancer, or esophagogastric cancer.

31. The method of any one of claims 1 to 30, wherein the PARP inhibitor is a compound of formula (III) or a pharmaceutically acceptable salt thereof.

32. The method of claim 30, wherein R³ in formula (III) is C₁₋₄ alkyl.

33. The method of claim 31, wherein R³ in formula (III) is methyl.

34. The method of any one of claims 31 to 33, wherein R¹ in formula (III) is ethyl.

35. The method of claim 31, wherein the PARP inhibitor is a compound of formula (IIIa): 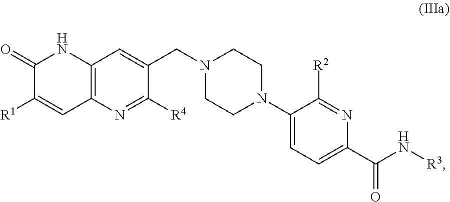
wherein
R¹ is C₁₋₄ alkyl,
R² is H, halo, C₁₋₄ alkyl, or C₁₋₄ fluoroalkyl,
R³ is H or C₁₋₄ alkyl, and
R⁴ is H,
or a pharmaceutically acceptable salt thereof.

36. The method of claim 35, wherein R² is difluoromethyl, trifluoromethyl, or methyl.

37. The method of claim 35, wherein R² is H or halo.

38. The method of claim 35, wherein R¹ is ethyl; R² is H, chloro or fluoro; and R³ is methyl.

39. The method of claim 31, wherein the PARP inhibitor is a compound of formula (IIIb): 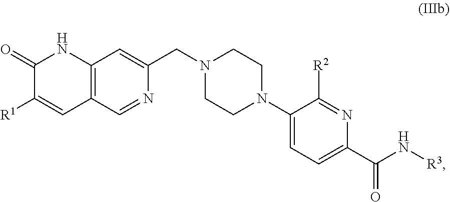
wherein
R¹ is C₁₋₄ alkyl,
R² is H or halo, and
R³ is H or C₁₋₄ alkyl,
or a pharmaceutically acceptable salt thereof.

40. The method of claim 39, wherein R¹ is ethyl; R² is H, chloro, or fluoro; and R³ is methyl.

41. The method of claim 31, wherein the PARP inhibitor is a compound of formula (IIIc): 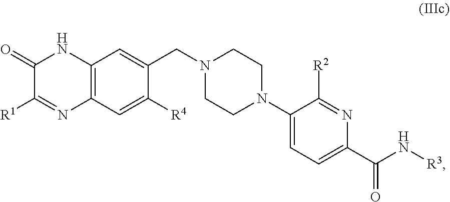
wherein
R¹ is C₁₋₄ alkyl or C₁₋₄ fluoroalkyl,
R² is H, halo, C₁₋₄ alkyl, or C₁₋₄ fluoroalkyl,
R³ is H or C₁₋₄ alkyl, and
R⁴ is H or fluoro,
or a pharmaceutically acceptable salt thereof.

42. The method of claim 41, wherein R¹ is ethyl, n-propyl, trifluoromethyl, 1,1-difluoroethyl, 2,2-difluroethyl, 2-fluoroethyl, or 2,2,2-trifluoroethyl.

43. The method of claim 41 or 42, wherein R² is H, methyl, ethyl, trifluoromethyl, difluoromethyl, fluoromethyl, fluoro, or chloro.

44. The method of any one of claims 41 to 43, wherein R³ is H or methyl.

45. The method of any one of claims 41 to 44, wherein R⁴ is H.

46. The method of any one of claims 1 to 30, wherein the PARP inhibitor is:
5-[4-[(3-ethyl-2-oxo-1H-1,6-naphthyridin-7-yl)methyl]piperazin-1-yl]-N-methyl-pyridine-2-carboxamide,
5-[4-[(3-ethyl-2-oxo-1H-1,6-naphthyridin-7-yl)methyl]piperazin-1-yl]-6-fluoro-N-methyl-pyridine-2-carboxamide,
6-chloro-5-[4-[(3-ethyl-2-oxo-1H-1,6-naphthyridin-7-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-6-fluoro-N-methyl-pyridine-2-carboxamide,
6-chloro-5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]pyridine-2-carboxamide,
6-ethyl-5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-N-methyl-6-(trifluoromethyl)pyridine-2-carboxamide,
6-(difluoromethyl)-5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-6-fluoro-N-methyl-pyridine-2-carboxamide,
5-[4-[(2-ethyl-3-oxo-4H-quinoxalin-6-yl)methyl]piperazin-1-yl]-N,6-dimethyl-pyridine-2-carboxamide,
6-chloro-5-[4-[(2-ethyl-3-oxo-4H-quinoxalin-6-yl)methyl]piperazin-1-yl]-N-methyl-pyridine-2-carboxamide,
N-methyl-5-[4-[[3-oxo-2-(trifluoromethyl)-4H-quinoxalin-6-yl]methyl]piperazin-1-yl]pyridine-2-carboxamide,
6-chloro-N-methyl-5-[4-[[3-oxo-2-(trifluoromethyl)-4H-quinoxalin-6-yl]methyl]piperazin-1-yl]pyridine-2-carboxamide,
6-fluoro-N-methyl-5-[4-[[3-oxo-2-(trifluoromethyl)-4H-quinoxalin-6-yl]methyl]piperazin-1-yl]pyridine-2-carboxamide,
N-methyl-5-[4-[(3-oxo-2-propyl-4H-quinoxalin-6-yl)methyl]piperazin-1-yl]pyridine-2-carboxamide,
6-chloro-N-methyl-5-[4-[(3-oxo-2-propyl-4H-quinoxalin-6-yl)methyl]piperazin-1-yl]pyridine-2-carboxamide,
6-fluoro-N-methyl-5-[4-[(3-oxo-2-propyl-4H-quinoxalin-6-yl)methyl]piperazin-1-yl]pyridine-2-carboxamide,
5-[4-[(2-ethyl-7-fluoro-3-oxo-4H-quinoxalin-6-yl)methyl]piperazin-1-yl]-6-fluoro-N-methyl-pyridine-2-carboxamide,
5-[4-[[2-(1,1-difluoroethyl)-3-oxo-4H-quinoxalin-6-yl]methyl]piperazin-1-yl]-N-methyl-pyridine-2-carboxamide,
5-[4-[[2-(2,2-difluoroethyl)-3-oxo-4H-quinoxalin-6-yl]methyl]piperazin-1-yl]-N-methyl-pyridine-2-carboxamide,
5-[4-[[2-(2,2-difluoroethyl)-3-oxo-4H-quinoxalin-6-yl]methyl]piperazin-1-yl]-6-fluoro-N-methyl-pyridine-2-carboxamide,
5-[4-[[2-(2-fluoroethyl)-3-oxo-4H-quinoxalin-6-yl]methyl]piperazin-1-yl]-N-methyl-pyridine-2-carboxamide,
6-fluoro-5-[4-[[2-(2-fluoroethyl)-3-oxo-4H-quinoxalin-6-yl]methyl]piperazin-1-yl]-N-methyl-pyridine-2-carboxamide,
N-methyl-5-[4-[[3-oxo-2-(2,2,2-trifluoroethyl)-4H-quinoxalin-6-yl]methyl]piperazin-1-yl]pyridine-2-carboxamide,
6-fluoro-N-methyl-5-(4-((3-oxo-2-(2,2,2-trifluoroethyl)-3,4-dihydroquinoxalin-6-yl)methyl) piperazin-1-yl)picolinamide,
or a pharmaceutically acceptable salt thereof.

47. The method of any one of claims 1 to 30, wherein the PARP inhibitor is 5-[4-[(7-ethyl-6-oxo-5H-1,5-naphthyridin-3-yl)methyl]piperazin-1-yl]-N-methyl-pyridine-2-carboxamide, or a pharmaceutically acceptable salt thereof.

48. The method of any one of claims 1 to 30, wherein the PARP inhibitor is:
6-(difluoromethyl)-5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-N-methyl-6 (trifluoromethyl)pyridine-2-carboxamide,
5-[4-[(7-ethyl-6-oxo-5H-1,5-naphthyridin-3-yl)methyl]piperazin-1-yl]-N,6-dimethyl-pyridine-2-carboxamide,
N-ethyl-5-[4-[(7-ethyl-6-oxo-5H-1,5-naphthyridin-3-yl)methyl]piperazin-1-yl]pyridine-2-carboxamide,
or a pharmaceutically acceptable salt thereof.

49. The method of any one of claims 1 to 30, wherein the PARP inhibitor is 5-[4-[(7-ethyl-6-oxo-5H-1,5-naphthyridin-3-yl)methyl]piperazin-1-yl]-N-methyl-pyridine-2-carboxamide or a pharmaceutically acceptable salt thereof.

50. The method of any one of claims 1 to 30, wherein the PARP inhibitor is a compound of formula (IV) or a pharmaceutically acceptable salt thereof.

51. The method of claim 50, wherein R¹ is methyl, ethyl, isopropyl, cyclopropyl, 1,1-difluoroethyl, 1-fluoroethyl, trifluoromethyl, difluoromethyl, or methoxy.

52. The method of claim 51, wherein R¹ is methyl or ethyl.

53. The method of any one of claims 50 to 52, wherein R² is H, chloro, fluoro, methyl, or difluoromethyl.

54. The method of claim 53, wherein R² is fluoro or methyl.

55. The method of any one of claims 50 to 54, wherein R³ is methyl or ethyl.

56. The method of any one of claims 50 to 55, wherein R⁴ is chloro, fluoro, or methyl.

57. The method of claim 56, wherein R⁴ is fluoro.

58. The method of claim 50, wherein R¹ is C₁₋₄ alkyl, R² is halo, R³ is C₁₋₄ alkyl, and R⁴ is halo or C₁₋₄ alkyl.

59. The method of any one of claims 1 to 30, wherein the PARP inhibitor is:
5-[4-[(2,5-dimethyl-3-oxo-4H-quinoxalin-6-yl)methyl]piperazin-1-yl]-6-fluoro-N-methyl-pyridine-2-carboxamide, 5-[4-[(2,5-dimethyl-3-oxo-4H-quinoxalin-6-yl)methyl]piperazin-1-yl]-N-methyl-pyridine-2-carboxamide,
6-chloro-5-[4-[(2,5-dimethyl-3-oxo-4H-quinoxalin-6-yl)methyl]piperazin-1-yl]-N-methyl-pyridine-2-carboxamide,
5-[4-[(2,5-dimethyl-3-oxo-4H-quinoxalin-6-yl)methyl]piperazin-1-yl]-N,6-dimethyl-pyridine-2-carboxamide,
5-[4-[(2,5-dimethyl-3-oxo-4H-quinoxalin-6-yl)methyl]piperazin-1-yl]-6-fluoro-pyridine-2-carboxamide,
5-[4-[(5-fluoro-2-methyl-3-oxo-4H-quinoxalin-6-yl)methyl]piperazin-1-yl]-6-methyl-pyridine-2-carboxamide,
5-[4-[(2,5-dimethyl-3-oxo-4H-quinoxalin-6-yl)methyl]piperazin-1-yl]-6-methyl-pyridine-2-carboxamide,
6-chloro-5-[4-[(5-chloro-2-ethyl-3-oxo-4H-quinoxalin-6-yl)methyl]piperazin-1-yl]-N-methyl-pyridine-2-carboxamide,
5-[4-[(5-chloro-2-ethyl-3-oxo-4H-quinoxalin-6-yl)methyl]piperazin-1-yl]-6-fluoro-N-methyl-pyridine-2-carboxamide,
5-[4-[(5-chloro-2-ethyl-3-oxo-4H-quinoxalin-6-yl)methyl]piperazin-1-yl]-N-methyl-pyridine-2-carboxamide,
5-[4-[(5-chloro-2-ethyl-3-oxo-4H-quinoxalin-6-yl)methyl]piperazin-1-yl]-N,6-dimethyl-pyridine-2-carboxamide,
6-fluoro-5-[4-[[5-fluoro-2-[(1S and 1R)-1-fluoroethyl]-3-oxo-4H-quinoxalin-6-yl]methyl]piperazin-1-yl]-N-methyl-pyridine-2-carboxamide,
5-[4-[[5-fluoro-2-[(1S and 1R)-1-fluoroethyl]-3-oxo-4H-quinoxalin-6-yl]methyl]piperazin-1-yl]-N,6-dimethyl-pyridine-2-carboxamide,
5-[4-[(5-chloro-2-methyl-3-oxo-4H-quinoxalin-6-yl)methyl]piperazin-1-yl]-N-methyl-pyridine-2-carboxamide,
5-[4-[(5-chloro-2-methyl-3-oxo-4H-quinoxalin-6-yl)methyl]piperazin-1-yl]-6-fluoro-N-methyl-pyridine-2-carboxamide,
5-[4-[(5-chloro-2-methyl-3-oxo-4H-quinoxalin-6-yl)methyl]piperazin-1-yl]-N,6-dimethyl-pyridine-2-carboxamide,
5-[4-[[2-(1,1-difluoroethyl)-5-fluoro-3-oxo-4H-quinoxalin-6-yl]methyl]piperazin-1-yl]-N, 6-dimethyl-pyridine-2-carboxamide,
6-fluoro-5-[4-[(5-fluoro-2-methyl-3-oxo-4H-quinoxalin-6-yl)methyl]piperazin-1-yl]-N-methyl-pyridine-2-carboxamide, 6-(difluoromethyl)-5-[4-[(5-fluoro-2-methyl-3-oxo-4H-quinoxalin-6-yl)methyl]piperazin-1-yl]-N-methyl-pyridine-2-carboxamide,
6-fluoro-5-[4-[(5-fluoro-2-methyl-3-oxo-4H-quinoxalin-6-yl)methyl]piperazin-1-yl]pyridine-2-carboxamide,
5-[4-[(2-ethyl-5-fluoro-3-oxo-4H-quinoxalin-6-yl)methyl]piperazin-1-yl]-N,6-dimethyl-pyridine-2-carboxamide,
6-(difluoromethyl)-5-[4-[(2-ethyl-5-fluoro-3-oxo-4H-quinoxalin-6-yl)methyl]piperazin-1-yl]-N-methyl-pyridine-2-carboxamide,
5-[4-[(2-ethyl-5-fluoro-3-oxo-4H-quinoxalin-6-yl)methyl]piperazin-1-yl]pyridine-2-carboxamide,
5-[4-[(2-ethyl-5-fluoro-3-oxo-4H-quinoxalin-6-yl)methyl]piperazin-1-yl]-6-methyl-pyridine-2-carboxamide,
5-[4-[(2-ethyl-5-fluoro-3-oxo-4H-quinoxalin-6-yl)methyl]piperazin-1-yl]-6-fluoro-N-methyl-pyridine-2-carboxamide,
6-chloro-5-[4-[(2-ethyl-5-fluoro-3-oxo-4H-quinoxalin-6-yl)methyl]piperazin-1-yl]-N-methyl-pyridine-2-carboxamide,
5-[4-[(2-ethyl-5-fluoro-3-oxo-4H-quinoxalin-6-yl)methyl]piperazin-1-yl]-N-methyl-pyridine-2-carboxamide,
6-chloro-5-[4-[(5-fluoro-2-methyl-3-oxo-4H-quinoxalin-6-yl)methyl]piperazin-1-yl]-N-methyl-pyridine-2-carboxamide,
5-[4-[(5-fluoro-2-methyl-3-oxo-4H-quinoxalin-6-yl)methyl]piperazin-1-yl]-N,6-dimethyl-pyridine-2-carboxamide,
5-[4-[(5-fluoro-2-methyl-3-oxo-4H-quinoxalin-6-yl)methyl]piperazin-1-yl]-N-methyl-pyridine-2-carboxamide,
5-[4-[(5-fluoro-3-oxo-4H-quinoxalin-6-yl)methyl]piperazin-1-yl]-N-methyl-pyridine-2-carboxamide,
6-chloro-5-[4-[(5-fluoro-3-oxo-4H-quinoxalin-6-yl)methyl]piperazin-1-yl]-N-methyl-pyridine-2-carboxamide,
5-[4-[(5-fluoro-3-oxo-4H-quinoxalin-6-yl)methyl]piperazin-1-yl]-N,6-dimethyl-pyridine-2-carboxamide,
6-fluoro-5-[4-[(5-fluoro-3-oxo-4H-quinoxalin-6-yl)methyl]piperazin-1-yl]-N-methyl-pyridine-2-carboxamide,
5-[4-[[2-(difluoromethyl)-5-fluoro-3-oxo-4H-quinoxalin-6-yl]methyl]piperazin-1-yl]-N, 6-dimethyl-pyridine-2-carboxamide, 5-[4-[(5-fluoro-2-methoxy-3-oxo-4H-quinoxalin-6-yl)methyl]piperazin-1-yl]-N-methyl-pyridine-2-carboxamide,
6-fluoro-5-[4-[(5-fluoro-2-methoxy-3-oxo-4H-quinoxalin-6-yl)methyl]piperazin-1-yl]-N-methyl-pyridine-2-carboxamide,
5-[4-[(5-fluoro-2-methoxy-3-oxo-4H-quinoxalin-6-yl)methyl]piperazin-1-yl]-N,6-dimethyl-pyridine-2-carboxamide,
6-chloro-5-[4-[(5-fluoro-2-methoxy-3-oxo-4H-quinoxalin-6-yl)methyl]piperazin-1-yl]-N-methyl-pyridine-2-carboxamide,
5-[4-[(2-ethyl-5-methyl-3-oxo-4H-quinoxalin-6-yl)methyl]piperazin-1-yl]-N,6-dimethyl-pyridine-2-carboxamide,
5-[4-[(2-ethyl-5-methyl-3-oxo-4H-quinoxalin-6-yl)methyl]piperazin-1-yl]-6-fluoro-N-methyl-pyridine-2-carboxamide,
5-[4-[(2-ethyl-5-methyl-3-oxo-4H-quinoxalin-6-yl)methyl]piperazin-1-yl]-N-methyl-pyridine-2-carboxamide,
N-ethyl-6-fluoro-5-[4-[(5-fluoro-2-methyl-3-oxo-4H-quinoxalin-6-yl)methyl]piperazin-1-yl]pyridine-2-carboxamide,
N-ethyl-5-[4-[(5-fluoro-2-methyl-3-oxo-4H-quinoxalin-6-yl)methyl]piperazin-1-yl]-6-methyl-pyridine-2-carboxamide,
5-[4-[[5-fluoro-3-oxo-2-(trifluoromethyl)-4H-quinoxalin-6-yl]methyl]piperazin-1-yl]-N, 6-dimethyl-pyridine-2-carboxamide,
6-fluoro-5-[4-[[5-fluoro-3-oxo-2-(trifluoromethyl)-4H-quinoxalin-6-yl]methyl]piperazin-1-yl]-N-methyl-pyridine-2-carboxamide,
6-chloro-5-[4-[[5-fluoro-3-oxo-2-(trifluoromethyl)-4H-quinoxalin-6-yl]methyl]piperazin-1-yl]-N-methyl-pyridine-2-carboxamide,
5-[4-[[5-fluoro-3-oxo-2-(trifluoromethyl)-4H-quinoxalin-6-yl]methyl]piperazin-1-yl]-N-methyl-pyridine-2-carboxamide,
6-fluoro-5-[4-[(5-fluoro-2-isopropyl-3-oxo-4H-quinoxalin-6-yl)methyl]piperazin-1-yl]-N-methyl-pyridine-2-carboxamide,
5-[4-[(5-fluoro-2-isopropyl-3-oxo-4H-quinoxalin-6-yl)methyl]piperazin-1-yl]-N,6-dimethyl-pyridine-2-carboxamide,
5-[4-[(5-fluoro-2-isopropyl-3-oxo-4H-quinoxalin-6-yl)methyl]piperazin-1-yl]-N-methyl-pyridine-2-carboxamide, 5-[4-[(2-cyclopropyl-5-fluoro-3-oxo-4H-quinoxalin-6-yl)methyl]piperazin-1-yl]-6-fluoro-N-methyl-pyridine-2-carboxamide,
5-[4-[(2-cyclopropyl-5-fluoro-3-oxo-4H-quinoxalin-6-yl)methyl]piperazin-1-yl]-N,6-dimethyl-pyridine-2-carboxamide,
5-[4-[(2-cyclopropyl-5-fluoro-3-oxo-4H-quinoxalin-6-yl)methyl]piperazin-1-yl]-N-methyl-pyridine-2-carboxamide,
5-[4-[(2-methoxy-5-methyl-3-oxo-4H-quinoxalin-6-yl)methyl]piperazin-1-yl]-N,6-dimethyl-pyridine-2-carboxamide,
6-fluoro-5-[4-[(2-methoxy-5-methyl-3-oxo-4H-quinoxalin-6-yl)methyl]piperazin-1-yl]-N-methyl-pyridine-2-carboxamide,
6-(difluoromethyl)-5-[4-[(2-methoxy-5-methyl-3-oxo-4H-quinoxalin-6-yl)methyl]piperazin-1-yl]-N-methyl-pyridine-2-carboxamide,
6-(difluoromethyl)-5-[4-[(2,5-dimethyl-3-oxo-4H-quinoxalin-6-yl)methyl]piperazin-1-yl]-N-methyl-pyridine-2-carboxamide,
or a pharmaceutically acceptable salts thereof.

60. The method of any one of claims 1 to 30, wherein the PARP inhibitor is 6-fluoro-5-[4-[(5-fluoro-2-methyl-3-oxo-4H-quinoxalin-6-yl)methyl]piperazin-1-yl]-N-methyl-pyridine-2-carboxamide, or a pharmaceutically acceptable salt thereof.

61. The method of any one of claims 1 to 30, wherein the PARP inhibitor is 6-fluoro-5-[4-[(5-fluoro-2-methyl-3-oxo-4H-quinoxalin-6-yl)methyl]piperazin-1-yl]-N-methyl-pyridine-2-carboxamide.

62. A method of treating a cancer in a subject, the method comprising administering to the subject in need thereof a therapeutically effective amount of an ATR inhibitor and a therapeutically effective amount of a PARP inhibitor, wherein the cancer has been previously identified as a cancer having a loss of function of ATM, BRCA2, RNAse H2A, RNAse H2B, CDK12, or a combination thereof, or wherein the cancer has been previously identified as an ALT+ cancer, and wherein the PARP inhibitor is veliparib (ABT-888), iniparib (BSI-201), 2X-121, CEP-9722, KU-0059436 (AZD2281), PF-01367338, a pharmaceutically acceptable salt thereof, or a combination thereof.

63. A method of treating a cancer in a subject, the method comprising administering to the subject in need thereof a therapeutically effective amount of an ATR inhibitor and a therapeutically effective amount of a PARP inhibitor, wherein the cancer has a loss of function of ATM, BRCA2, RNAse H2A, RNAse H2B, CDK12, or a combination thereof, or wherein the cancer is an ALT+ cancer; and wherein the PARP inhibitor is veliparib (ABT-888), iniparib (BSI-201), 2X-121, CEP-9722, KU-0059436 (AZD2281), PF-01367338, a pharmaceutically acceptable salt thereof, or a combination thereof.

64. A method of treating a cancer in a subject, the method comprising:
(i) identifying the cancer as having a loss of function of ATM, BRCA2, RNAse H2A, RNAse H2B, CDK12, or a combination thereof, or as an ALT+ cancer; and
(ii) administering to the subject in need thereof a therapeutically effective amount of an ATR inhibitor and a therapeutically effective amount of a PARP inhibitor that is veliparib (ABT-888), iniparib (BSI-201), 2X-121, CEP-9722, KU-0059436 (AZD2281), PF-01367338, a pharmaceutically acceptable salt thereof, or a combination thereof.

65. The method of any one of claims 62 to 64, wherein the ATR inhibitor is administered before the PARP inhibitor.

66. The method of any one of claims 62 to 64, wherein the ATR inhibitor is administered after the PARP inhibitor.

67. The method of any one of claims 62 to 64, wherein the ATR inhibitor is co-administered with the PARP inhibitor.

68. The method of any one of claims 62 to 67, wherein the therapeutically effective amount is a subtherapeutic regimen of the ATR inhibitor.

69. The method of any one of claims 62 to 68, wherein the therapeutically effective amount is a subtherapeutic regimen of the PARP inhibitor.

70. The method of claim 68 or 69, wherein the subtherapeutic regimen comprises a starting dosage that is at least 50% less than the lowest standard starting dosage that is used for a monotherapy.

71. The method of any one of claims 68 to 70, wherein the subtherapeutic regimen comprises a maintenance dosage that is at least 50% less than the lowest standard maintenance dosage that is used for a monotherapy.

72. The method of claim 71, wherein the maintenance dosage comprises a first reduced dosage.

73. The method of claim 71 or 72, wherein the maintenance dosage comprises a second reduced dosage.

74. The method of any one of claims 71 to 73, wherein the maintenance dosage comprises a third reduced dosage.

75. The method of any one of claims 62 to 74, wherein the route of administration is an oral administration.

76. The method of any one of claims 62 to 75, wherein the ATR inhibitor is administered 1 day/week, 2 days/week, or 3 days/week.

77. The method of any one of claims 62 to 76, wherein the PARP inhibitor is administered 1 day/week, 2 days/week, 3 days/week, or 4 days/week.

78. A method of inducing cell death in an aberrant cancer cell having a loss of function of ATM, BRCA2, RNAse H2A, RNAse H2B, CDK12, or a combination thereof, or in an ALT+ cancer cell, the method comprising contacting the cell with an effective amount of an ATR inhibitor and an effective amount of a PARP inhibitor, the effective amounts being sufficient to induce cell death in the aberrant cancer cell; wherein the PARP inhibitor is veliparib (ABT-888), iniparib (BSI-201), 2X-121, CEP-9722, KU-0059436 (AZD2281), PF-01367338, a pharmaceutically acceptable salt thereof, or a combination thereof.

79. The method of any one of claims 62 to 78, wherein the loss of function is a loss of function of ATM.

80. The method of any one of claims 62 to 78, wherein the loss of function is a loss of function of RNAse H2A.

81. The method of any one of claims 62 to 78, wherein the loss of function is a loss of function of RNAse H2B.

82. The method of any one of claims 62 to 78, wherein the loss of function is a loss of function of CDK12.

83. The method of any one of claims 62 to 78, wherein the loss of function is a loss of function of BRCA2.

84. The method of any one of claims 62 to 78, wherein the cancer is an ALT+ cancer.

85. The method of any one of claims 62 to 84, wherein the ATR inhibitor is a compound of formula (I): 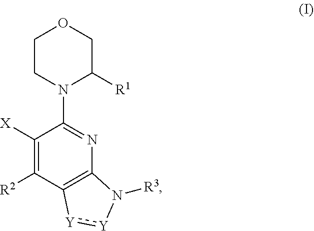
or a pharmaceutically acceptable salt thereof,
wherein [symbol] is a double bond, and each Y is independently N or CR⁴; or [symbol] is a single bond, and each Y is independently NR^{Y}, carbonyl, or C(R^{Y})₂; wherein each R^{Y} is independently H or optionally substituted C₁₋₆ alkyl;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

86. The method of claim 85, wherein the ATR inhibitor is a compound of formula (II): 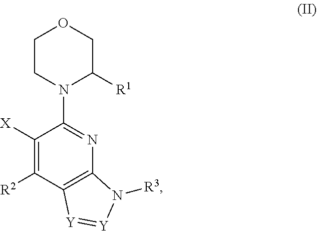
or a pharmaceutically acceptable salt thereof,
wherein
each Y is independently N or CR⁴;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

87. The method of claim 85, wherein the ATR inhibitor is selected from the group consisting of compounds 43, 57, 62, 87, 93, 94, 95, 99, 100, 106, 107, 108, 109, 111, 112, 113, 114, 115, 116, 118, 119, 120, 121, 122, 123, 135, 147, 148, and pharmaceutically acceptable salts thereof.

88. The method of claim 87, wherein the ATR inhibitor is compound 43 or a pharmaceutically acceptable salt thereof.

89. The method of claim 87, wherein the ATR inhibitor is compound 121 or a pharmaceutically acceptable salt thereof.

90. The method of claim 87, wherein the ATR inhibitor is compound 122 or a pharmaceutically acceptable salt thereof.
